SEQUENCE LISTING (8 sequences)

SEQ ID NO: 1 - DNA, 19 bp, Artificial Sequence
  NS1 Primer
gtagtcatatgcttgtctc

SEQ ID NO: 2 - DNA, 20 bp, Artificial Sequence
  NS8 Primer
tccgcaggttcacctacgga

SEQ ID NO: 3 - DNA, 19 bp, Artificial Sequence
  ITS1 Primer
tccgtaggtgaacctgcgg

SEQ ID NO: 4 - DNA, 20 bp, Artificial Sequence
  ITS4 Primer
tcctccgcttattgatatgc

SEQ ID NO: 5 - DNA, 20 bp, Artificial Sequence
  SP6 Primer
catttaggtgaacactatag

SEQ ID NO: 6 - DNA, 19 bp, Artificial Sequence
  T7 Primer
gtaatacgactcactatag

SEQ ID NO: 7 - DNA, 22 bp, Artificial Sequence
  4SP61 Primer
gcctttccttctggggagcatg

SEQ ID NO: 8 - DNA, 23 bp, Artificial Sequence
  4T71 Primer
ctgatcgtcttcgatcccctaac

(19) United States
(12) Patent Application Publication
(10) Pub. No.: US 2009/0142816 A1
(43) Pub. Date: Jun. 4, 2009

(54) GLIOCLADIUM ISOLATE C-13 AND METHODS OF ITS USE FOR PRODUCING VOLATILE COMPOUNDS AND HYDROCARBONS

(76) Inventor: Gary STROBEL, Bozeman, MT (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/262,663

(22) Filed: Oct. 31, 2008

Related U.S. Application Data
(60) Provisional application No. 60/984,234, filed on Oct. 31, 2007.
(60) Provisional application No. 60/986,609, filed on Nov. 9, 2007.
(60) Provisional application No. 61/085,172, filed on Jul. 31, 2008.
(60) Provisional application No. 61/096,913, filed on Sep. 15, 2008.

(51) Int. Cl. C12N 1/14; C12P 5/00; C12P 5/02; C12P 7/40; C12P 7/64; C12P 7/24; C12P 7/02; C07H 21/00; C12N 1/15

(52) U.S. Cl. 435/134; 435/254.1; 435/166; 435/167; 435/155; 435/147; 435/136; 435/256.8; 536/23.74; 435/254.11

(57) ABSTRACT

Provided herein is Gliocladium isolate C-13 (NRRL 50072) which is an isolated strain of a Gliocladium spp. obtained from an endophyte of a Eucryphia cordifolia plant. Methods of culturing Gliocladium isolate C-13 are provided. The methods can include culturing the Gliocladium isolate C-13 under conditions sufficient to produce hydrocarbons. Also disclosed are methods for producing a hydrocarbon, a hydrocarbon fuel and a blend useful as a fuel. For example, the hydrocarbons produced from isolate C-13 can be used to generate biofuels, such as biofuels for vehicles and aircraft. Also provided are kits which comprise Gliocladium isolate C-13 and methods of cloning, isolating, and expressing Gliocladium isolate C-13 genes.

### CROSS REFERENCE TO RELATED APPLICATIONS

This application claims priority to U.S. Provisional Application Ser. No. 60/984,234, filed Oct. 31, 2007, U.S. Provisional Application Ser. No. 60/986,609, filed Nov. 9, 2007, U.S. Provisional Application Ser. No. 61/085,172, filed Jul. 31, 2008, and U.S. Provisional Application Ser. No. 61/096,913, filed Sep. 15, 2008, each of which are herein incorporated by reference in their entireties for all purposes.

### ACKNOWLEDGEMENT OF GOVERNMENTAL SUPPORT

This invention was made with government support under CHE-0114469 awarded by the National Science Foundation. The government has certain rights in the invention.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: MONT-098_O3US.txt, date recorded: Oct. 31, 2008 file size 2 kilobytes).

### FIELD

The present disclosure relates to endophytic fungi, and in particular to the endophytic Gliocladium isolate C-13 of Eucryphia cordifolia, and methods of using Gliocladium isolate C-13 for producing selective volatile antimicrobial compounds and hydrocarbons.

### BACKGROUND

Bioprospecting is a term coined recently to refer to the search for novel products or organisms of economic importance from the world's biota. The notion exists that tropical forests are more species-rich than temperate forests, or arid forests and that within tropical regions the greatest microbial diversity is to be found. Therefore intensive sampling of unique habitats in a defined area will aide in the discovery of the undescribed fungi (Hawksworth and Rossman, Phytopathology 87:888-891, 1987). The 300,000 species of vascular plants seem to be serving as a reservoir of untold numbers of microbes known as endophytes (Bacon and White, Microbial Endophytes, Marcel Deker Inc., NY, 2000).

Endophytes, microorganisms that reside in the tissues of living plants (Stone et al., Microbial Endophytes, Ed. C. W. Bacon and J. F. White Marcel Decker, Inc, NY, 2000), are relatively unstudied and potential sources of novel natural products for exploitation in medicine, agriculture and industry. It is worthy to note, that of the nearly 300,000 plant species that exist on the earth, each individual plant is host to one or more endophytes. Only a handful of these plants have ever been completely studied relative to their endophytic biology. Consequently, the opportunity to find new and interesting endophytic microorganisms among myriads of plants in different settings, and ecosystems is great.

Endophytes are microbes that inhabit such biotopes, namely higher plants, which is why they are currently considered as a wellspring of novel secondary metabolites offering the potential for medical, agricultural and/or industrial exploitation. Currently, endophytes are viewed as an outstanding source of bioactive natural products because there are so many of them occupying literally millions of unique biological niches (higher plants) growing in so many unusual environments.

Recently, two endophytic fungi, isolated from monsoonal and tropical rainforests, were reported to produce volatile antibiotics. Muscodor roseus was isolated from two monsoonal rainforest tree species in Northern Australia (Worapong et al., Mycotaxon. 81: 463-475, 2001), while Muscodor albus was obtained from Cinnamomum zeylanicum in Honduras (Worapong et al., Mycotaxon. 79: 67-79, 2001). These endophytes produce a mixture of volatile antimicrobials that effectively inhibit and kill a wide spectrum of plant associated fungi and bacteria (Strobel et al., Microbiology 147: 2943-2950, 2001). Thus, while many wood inhabiting fungi make volatile metabolites including cyanide and cyano-like compounds, until now little practical value has been placed on them as potential biocontrol agents for use in agriculture, industry or medicine (McAfee and Taylor, Natural Toxins 7: 283-303, 1999). This is probably because none, except for the Muscodor spp., make complex mixtures of organic substances that have both a potent and selective antibiotic effect (Strobel et al., Microbiology 147: 2943-2950, 2001; McAfee and Taylor, Natural Toxins 7: 283-303, 1999).

Since the successful isolation of M. albus and M. roseus, the first volatile antibiotic producing endophytes reported, another Muscodor sp., including most recently, M. vitigenus, has been identified (Worapong et al., Mycotaxon. 81: 463-475, 2001; Worapong et al., Mycotaxon. 79: 67-79, 2001; Strobel et al., Microbiology 147: 2943-2950, 2001; Daisy et al., Microbiology 148: 3737-3741, 2002). M. vitigenus primarily produces biologically active amounts of naphthalene in culture and thus, can possibly be used as an insect deterrent. Although these studies have identified volatile antibiotic endophytes, all of them are endophytic fungi from the Muscodor spp. Therefore, non-Muscodor spp. endophytic fungi capable of producing volatile antibiotics remain to be identified. Additionally, endophytic organisms capable of producing products that could be used in industrial applications, such as the generation of hydrocarbons for use in biofuels, remain to be discovered.

### SUMMARY OF THE INVENTION

In one aspect, this invention provides an isolated strain of Gliocladium spp. In an exemplary embodiment, the isolated strain is Gliocladium isolate C-13 (deposited as NRRL 50072).

In another aspect, this invention provides a method for producing volative organic compounds (VOCs), comprising culturing Gliocladium isolate C-13 (NRRL 50072) under conditions sufficient for producing VOCs. In one embodiment, Gliocladium isolate C-13 is cultured in culture medium comprising oatmeal agar for the production of VOCs. In another embodiment, Gliocladium isolate C-13 is cultured in culture medium comprising oatmeal agar in microaerophilic conditions for the production of VOCs.

In some embodiments, Gliocladium isolate C-13 is cultured in a bioreactor vessel for the production of VOCs. In certain sub-embodiments, Gliocladium isolate C-13 is cultured in a bioreactor vessel having a volume from about 100 ml to about 10,000 L or larger. The VOCs are isolated from the culture medium or from the vapour in the vessel using several methods. In an exemplary embodiment, the VOCs are isolated using fractional distillation and/or absorption chromatography.

In some aspects, the VOC is an alkane, an alkene, an alkyne, a diene, an isoprene, an alcohol, an aldehyde, a carboxylic acid, a wax ester, or a mixture of any two or more thereof. In certain exemplary embodiments, the VOC is a compound found in Table 4, 7, 8, or 9. The VOCs of the invention can be used to produce a number of useful compositions, including, but not limited to biofuels, jet fuels, plastics, plasticizers, antibiotics, rubbers, fuel additives, and/or adhesives.

In another aspect, the invention provides a kit for making VOCs comprising Gliocladium spp. and instructions for growing said Gliocladium spp. under optimal conditions for VOC production. In an exemplary embodiment, the Gliocladium spp. is Gliocladium isolate C-13 (deposited as NRRL 50072). The kit may further comprise growth media, such as an oatmeal based media. In some embodiments, the Gliocladium spp. of the kit may be supplied frozen in media, freeze dried and/or as spores.

In another aspect, the invention provides an isolated strain of a Gliocladium such as isolate C-13, wherein the Gliocladium isolate has been serially propagated to change the metabolic characteristics and/or genetic make-up of the isolate. In certain embodiments, such changes increase or decrease the production of a compound(s) found in Tables 4, 7, 8, or 9.

In another aspect, the invention provides a method for producing VOCs comprising culturing an anamorph of Ascocoryne spp. under conditions sufficient for producing VOCs. In an exemplary embodiment, the anamorph of Ascocoryne spp. is Gliocladium isolate C-13.

In another aspect, the invention provides an isolated nucleic acid molecule from Gliocladium isolate C-13 (NRRL 50072) encoding a polypeptide involved in the synthesis or production of VOCs. In one embodiment, said VOC is a hydrocarbon. In another embodiment, said hydrocarbon is selected from the group consisting of 1,3,5,7,-cyclooctatetraene, 1-octene, 1,3 octadiene, 7-octen-4-ol. In another embodiment, said nucleic acid molecule is cloned into a vector. In yet another embodiment, said vector is transformed or transfected into a heterologous cell.

The invention also provides a chromosomal library of Gliocladium isolate C-13 (NRRL 50072). In one embodiment, said library is cloned into a vector that can replicate in a prokaryotic cell or fungus. In another embodiment, said library is a lambda phage, YAC, BAC, and/or cDNA. In another embodiment, said library is screened for production of VOCs. In yet another embodiment, said VOC is a hydrocarbon.

In yet another aspect, the invention provides an isolated nucleic acid molecule, wherein said isolated nucleic acid molecule is at least 65%, at least 70%, at least 75%, at least 80%, at least 85%, at least 90%, at least 95%, at least 96%, at least 97%, at least 98% or at least 99% identical to said isolated nucleic acid molecule from Gliocladium isolate C-13 (NRRL 50072). In one embodiment, said isolated nucleic acid molecule encodes for a polypeptide involved in the synthesis or production of VOCs. In another embodiment, said polypeptide is at least 65%, at least 70%, at least 75%, at least 80%, at least 85%, at least 90%, at least 95%, at least 96%, at least 97%, at least 98% or at least 99% identical to a polypeptide from Gliocladium isolate C-13.

The invention also provides an isolated nucleic acid molecule from Gliocladium isolate C-13, wherein Gliocladium isolate C-13 (deposited as NRRL 50072) was serially propagated, thereby changing the metabolic characteristic and/or genetic make-up of said Gliocladium isolate C-13. In one embodiment, said genetic make-up alteration increases and/or decreases the production of a compound(s) found in Tables 4, 7, 8, or 9.

In another aspect, the invention provides a vector comprising the isolated nucleic acid molecule from Gliocladium isolate C-13 (NRRL 50072) encoding a polypeptide involved in the synthesis or production of VOCs. In one embodiment, said VOC is a hydrocarbon.

The invention also provides a heterologous organism comprising the isolated nucleic acid molecule from Gliocladium isolate C-13 (NRRL 50072) encoding a polypeptide involved in the synthesis or production of VOCs. In one embodiment, said VOC is a hydrocarbon.

### BRIEF DESCRIPTION OF THE DRAWINGS

FIG. 1A is digital image illustrating the leaves and stems of Eucryphia cordifolia, the source plant of Gliocladium sp. obtained from the northern Patagonian region of Chile.

FIG. 1B is a digital image of a 14 day old culture of Gliocladium sp. growing on (Potato Dextrose Agar) PDA. Small dark spherical-like objects are present at the edge of the colony.

FIGS. 2A-2D are scanning electron micrographs of Gliocladium sp., Bar, 10 μm. FIG. 2A is a scanning electron micrograph of a young culture of Gliocladium sp. growing on PDA. FIG. 2B is a scanning electron micrograph of a colony colonizing carnation leaf tissue. FIG. 2C is a scanning electron micrograph of phialides with hyphal cells and conidiospores in the background. FIG. 2D is a scanning electron micrograph of conidiospores at a higher magnification.

FIG. 3 is an illustration of the chemical structure of 1,3,5,7-cyclooctatetraene, or [8]annulene.

FIG. 4 is an illustration of total ion production as measured by PTR-MS in the air space of an 18 day old culture of G. roseum on oatmeal-based medium. The ions produced from the agar alone are shown on the left while those of the fungus plus the agar are on the right. Ions that were deemed as reagent ions, contaminant ions such as O₂+ or NO+, or water-related ions were not included in this calculation.

FIG. 5 is an illustration of PTR-MS mass spectrum generated from the air space over an 18 day old culture of G. roseum grown at 23° C. on oatmeal medium. Prominent ions associated with the volatile components produced by G. roseum were identified. Ions indicated with an asterisk represent primary or impurity reagent ions, H3O+(H₂O)ₙ, NO+, O₂+, and not included in the calculation of the total gas concentration. The ions observed at masses over 200 amu (atomic mass units or Daltons) are associated with the agar medium.

### DETAILED DESCRIPTION

To investigate the biological phenomena of volatile antibiotic producing fungi, it was deemed important to learn if fungi other than Muscodor spp. produce them, to study the components of their volatiles, and to ascertain the breadth and scope of their biological activities. A cursory search of the endophytic fungi of representative Gondwanaland tree species was conducted in the area from the northern to the southern tip of the Patagonian region of South America. This region was picked because of the extremely ancient association of many tree species here to a time when the Gondwanaland existed about 100 million years ago. The rationale for this approach is that long held associations of plants with their respective landscapes have had an enormous time frame in which to form interactions with microorganisms in their respective environments.

Provided herein is an endophytic Gliocladium sp. associated with a Gondwanaland tree genus Eucryphia cordifolia. In an exemplary embodiment, the invention provides a specific Gliocladium sp. isolate, referred to as isolate C-13 (deposited as NRRL 50072). The disclosed Gliocladium isolate C-13 can also be classified as an endophytic Trichoderma sp. For a review of the fungi belonging to the genera Trichoderma and Gliocladium see Trichoderma and Gliocladium Volume 1: Basic biology, taxonomy and genetics, C. P. Kubicek and Gary E. Harman (editors), July 1998 (ISBN-10: 0748405720; CRC, 1^{st} Edition, 300 pages) and Trichoderma and Gliocladium, C. P. Kubicek and Gary E. Harman (editors), June 1998 (ISBN-10: 0748408053; CRC, 1^{st} Edition, 300 pages), both of which are incorporated herein by reference in their entireties.

The present inventor has further characterized the Gliocladium C-13 isolate via molecular techniques. The 5.8S, ITS1 and ITS2 regions of the organism were isolated, cloned, partially sequenced and deposited in GenBank as AY219041. In addition, the partial 18S rDNA sequence was entered as AY219040. Using a BLAST search, as a close relative, Ascocoryne cylichnium appears with a coverage of 90% and an identity of 98%. On the other hand, Ascocoryne sarcoides had a coverage of 89% and an identity of 99%. Comparative molecular genetics, via a phylogenetic tree, indicate that A. sarcoides is closely related to this fungus. A. sarcoides is an ascomycete whose anamorph does not have a listing of Gliocladium sp. The teleomorphs of Gliocladium spp. are generally considered as Nectria spp. and Hypocrea spp. (Handlin, R. T., In Illustrated Genera of Ascomycetes, Am Phytopath Press, St. Paul, Minn., 1989). Nevertheless, it is distinctly possible that this organism, as an atypical Gliocladium roseum, exists as an anamorph of Ascocoryne spp.

Therefore, in some embodiments, the anamorph forms of Ascocoryne spp. are contemplated for the generation of volatile organic compounds as described herein. Known anamorphs of Ascocoryne sarcoides include Coryne dubia and Phialophora spp. The metabolism of the anamorph renders it particularly suitable for a high degree of expression. A teleomorph should also be suitable as the genetic make-up of the anamorphs and teleomorphs is similar. The difference between the anamorph and teleomorph is that one is the asexual state and the other is the sexual state; the two states exhibit different morphology under certain conditions. In cases where fungi reproduce both sexually and asexually, these fungi have two names: the teleomorph name describes the fungus when reproducing sexually; the anamorph name refers to the fungus when reproducing asexually. The holomorph name refers to the “whole fungus”, encompassing both reproduction methods. When referring to one of these names in this invention, the “whole” fungus is referred to.

Synonyms of A. sarcoides include, but are not limited to, Ombrophilia sarcoides, Bulgaria sarcoides, Coryne sarcoides, Helvella sarcoides, Pirobasidium sarcoides, Tremella sarcoides, Scleroderris majuscula, Peziza sarcoides, and Lichen sarcoides.

It will be understood that for the aforementioned Gliocladium spp. and Ascocoryne spp. and synonyms thereof, the invention encompasses both the perfect and imperfect (“anamorph”) states, and other taxonomic equivalents, e.g., teleomorphs, regardless of the species name by which they are known. Those skilled in the art will readily recognize the identity of appropriate equivalents.

The present inventor has found that Gliocladium isolate C-13 possesses inhibitory activity against certain plant pathogenic fungi. For example, the gases of Gliocladium isolate C-13 can be used to inhibit the growth of Pythium ultimum, Gliocladium virens, Sclerotinia sclerotiorum, Verticillium dahaliae, Rhizoctonia solani, Geotrichum candidum, and Aspergillus ochraceus (as described in the examples provided herein). Thus, in one aspect, the invention provides compositions made from or generated by Gliocladium isolate C-13 that have bioactivity against one or more pathogenic fungi.

The present inventor has also found that endophytic Gliocladium sp. of Eucryphia cordifolia are involved in the biosynthesis of volatile organic compounds (VOCs) (e.g. hydrocarbons). Thus, in one embodiment, Gliocladium isolate C-13 can be used as a renewable energy source or “RES” for various hydrocarbon products. Such a novel, renewable source of hydrocarbons is desirable because it provides a supplement to the existing limited resources of non-renewable hydrocarbons. In addition, it permits the production of a wide range of specific hydrocarbon products designed for particular applications. For instance, specific hydrocarbon products can be produced and utilized to form biofuels.

Renewable energy sources or “RES” refer generally to energy sources that capture their energy from existing flows of energy, from on-going natural processes, such as biological processes. Most renewable forms of energy, other than geothermal and tidal power, ultimately come from the sun. The energy in biomass is accumulated over a period of months, as in straw, or through many years as in wood. Capturing renewable energy by plants, animals and humans does not permanently deplete the resource. Renewable energy resources may be used directly, or used indirectly to create other more convenient forms of energy. Examples of indirect use which require energy harvesting include production of fuels, such as ethanol, from biomass.

In one aspect, the present invention provides a method for producing volatile organic compounds (VOCs) (e.g. hydrocarbons). In one embodiment, the method comprises culturing Gliocladium isolate C-13 (NRRL 50072) under conditions sufficient for producing VOCs. As used herein, the phrase “volatile organic compound(s) (VOCs)” refers generally to organic chemical compounds that have high enough vapor pressures under normal conditions to significantly vaporize and enter the atmosphere. A wide range of carbon-based molecules, such as aldehydes, ketones, and hydrocarbons are VOCs. VOCs comprise the term “hydrocarbon” and “hydrocarbon product.” They are generally used interchangeably herein.

As used herein, the term “hydrocarbon” generally refers to a chemical compound that consists of the elements carbon (C) and hydrogen (H). All hydrocarbons consist of a carbon backbone and atoms of hydrogen attached to that backbone. Sometimes, the term is used as a shortened form of the term “aliphatic hydrocarbon.” There are essentially three types of hydrocarbons: (1) aromatic hydrocarbons, which have at least one aromatic ring; (2) saturated hydrocarbons, also known as alkanes, which lack double, triple or aromatic bonds; and (3) unsaturated hydrocarbons, which have one or more double or triple bonds between carbon atoms, and are divided into: alkenes, alkynes, and dienes. Liquid geologically-extracted hydrocarbons are generally referred to as petroleum (literally “rock oil”) or mineral oil, while gaseous geologic hydrocarbons are generally referred to as natural gas. All are significant sources of fuel and raw materials as a feedstock for the production of organic chemicals and are commonly found in the Earth's subsurface using the tools of petroleum geology. Oil reserves in sedimentary rocks are the principal source of hydrocarbons for the energy and chemicals industries. Hydrocarbons are of prime economic importance because they encompass the constituents of the major fossil fuels (coal, petroleum, natural gas, etc.) and biofuels, as well as plastics, waxes, solvents and oils.

A “hydrocarbon product,” as used herein, refers generally to a chemical compound that consists of the elements carbon (C), oxygen (O) and hydrogen (H). There are essentially three types of hydrocarbon products: (1) aromatic hydrocarbon products, which have at least one aromatic ring; (2) saturated hydrocarbon products, which lack double, triple or aromatic bonds; and (3) unsaturated hydrocarbon products, which have one or more double or triple bonds between carbon atoms. A “hydrocarbon product” may be further defined as a chemical compound that consists of C, H, and O with a carbon backbone and atoms of hydrogen and oxygen, attached to it. Oxygen may be singly or double bonded to the backbone and may be bound by hydrogen. In the case of ethers and esters, oxygen may be incorporated into the backbone, and linked by two single bonds, to carbon chains. A single carbon atom may be attached to one or more oxygen atoms. Hydrocarbon products may also include the above compounds attached to biological agents including proteins, coenzyme A and acetyl coenzyme A. Hydrocarbon products include, but are not limited to, hydrocarbons, alcohols, aldehydes, carboxylic acids, ethers, esters, and ketones.

The present disclosure also relates to methods for producing a biological agent, including an endophytic Gliocladium sp. with the desired characteristics. In one embodiment, the method includes cultivating a strain of an endophytic Gliocladium sp. under aerobic conditions on a medium including potato dextrose agar. The method can also include recovering biological compounds produced by the organism. Optionally, it may be desirable thereafter to form the free acid or a salt or ester of the biological compounds by methods known to those of ordinary skill.

As described above, the present invention provides a method for culturing Gliocladium isolate C-13 (NRRL 50072) under conditions sufficient for producing VOCs. In one embodiment, conditions sufficient for producing VOCs include culturing Gliocladium isolate C-13 (NRRL 50072) in culture medium comprising oatmeal, barley, or potato agar bases. The culture medium may also be PDA medium, a cellulose medium, or an Eucryphia cordifolia “ulmo” stem medium. In an exemplary embodiment, the culture medium comprises oatmeal agar. In some embodiments, these conditions can also include culturing Gliocladium isolate C-13 (NRRL 50072) in the absence of oxygen (anaerobic conditions) or in reduced oxygen conditions (e.g., microaerophilic conditions). In certain embodiments, the oxygen levels contain 20 percent or less the oxygen level than those levels present in the Earth's normal sea-level atmosphere. In certain exemplary embodiments, microaerophilic conditions include those in which the atmosphere has a 5 to 15 percent oxygen concentration as compared to those measured in the Earth's normal sea-level atmosphere. As noted below, the microaerophilic conditions may be favorable to the production of reduced compounds such as alkanes and cyclic alkanes.

Methods for growing microorganisms in microaerophilic conditions are generally well known in the art. For example, U.S. Pat. Nos. 4,562,051; 4,976,931; 5,955,344; 5,830,746; 6,204,051 and 6,429,008, each of which is hereby incorporated by reference in its entirety, provide apparatuses and methods for the generation of an anaerobic or microaerophilic atmosphere conducive to the growth of certain microorganisms. U.S. Pat. No. 4,377,554, which is hereby incorporated by reference in its entirety, provides gas generating devices for use in applications requiring a microaerophilic atmosphere.

In certain embodiments, Gliocladium isolate C-13 is cultured in a bioreactor vessel for the production of VOCs. Several methods of culturing Gliocladium in a bioreactor vessel can be employed to generate VOCs (e.g. hydrocarbons) and other desired molecules from Gliocladium isolate C-13 (NRRL 50072). For example, methods of culturing Gliocladium can include those disclosed in U.S. Pat. Nos. 7,232,908, 6,608,185, 6,511,821, 6,350,604, 5,407,826, 5,334,517, and 5,268,173, each of which is hereby incorporated by reference in its entirety. Any conventional bioreactor vessel can be used as the vessel for the purpose of this invention. The vessel may be made of materials such as stainless steel, glass, plastic, and/or ceramics, and may have a volume of from about 100 ml to 10,000 L or larger. The bioreactor vessel may be connected to a series of storage flasks that contain nutrient solutions and solutions for maintaining and controlling a desired pH and other parameters, such as foam formation, redox potential, etc., in the fermentation broth. Depending on the particular needs of the fermentation, there may be separate storage flasks for individual supply of substrates to the vessel, which substrates serve as the carbon, nitrogen or mineral source for the living cells in the vessel.

Several methods can be used to grow Gliocladium isolate C-13 for use in the invention. Fed Batch culture is a variation on ordinary batch culture and involves the addition of a nutrient feed to the batch. Cells are cultured in a medium in a fixed volume. Before the maximum cell concentration is reached, specific supplementary nutrients are added to the culture. The volume of the feed is minimal compared to the volume of the culture. Fed batch culture typically proceeds in a substantially fixed volume, for a fixed duration, and with a single harvest either when the cells have died or at an earlier, predetermined point.

In a continuous culture, the cells are initially grown in a fixed volume of medium. To avoid the onset of the decline phase, fresh medium is pumped into the bioreactor before maximum cell concentration is reached. The spent media, containing a proportion of the cells, is continuously removed from the bioreactor to maintain a constant volume. The process also removes the desired product, which can be continuously harvested, and provides a continuous supply of nutrients, which allows the cells to be maintained in an exponentially growing state. Theoretically, the process can be operated indefinitely. Continuous culture is characterized by a continuous increase in culture volume, an increase and dilution of the desired product, and continuous maintenance of an exponentially growing culture. There is no death or decline phase.

Perfusion culture is similar to continuous culture except that, when the medium is pumped out of the reactor, cells are not removed. As with a continuous culture, perfusion culture is an increasing-volume system with continuous harvest that theoretically can continue indefinitely.

Once produced, several methods can be used to isolate the VOCs (e.g. hydrocarbon such as alkanes, alkenes, alcohols, carboxylic acids and other hydrocarbons listed in Tables 4, 7, 8, and 9 below) from the culture medium or from the vapor in the growth chamber. For example, common separation techniques can be used to remove the cells from the broth, and common isolation procedures (e.g., extraction, distillation, and ion-exchange procedures) can be used to obtain VOC from the cell-free broth. See, U.S. Pat. Nos. 4,275,234, 5,510,526; 5,641,406, and 5,831,122, and International Patent Application Number WO 93/00440, each of which is hereby incorporated by reference in its entirety

Fractional distillation and/or absorption chromatography are non-limiting examples of methods to extract the desired product produced by Gliocladium isolate C-13. Fractional distillation is the separation of a mixture into its component parts, or fractions, such as in separating chemical compounds by their boiling point by heating them to a temperature at which several fractions of the compound will evaporate. It is a special type of distillation. Generally the component parts boil at less than 25° C. from each other under a pressure of one atmosphere (ATM). If the difference in boiling points is greater than 25° C., a simple distillation is used. Processes for fractional distillation are described in U.S. Pat. Nos. 4,405,449, 4,601,739, and 6,348,137, each of which is hereby incorporated by reference in its entirety.

Absorption chromatography is a physical separation method in which the components of a mixture are separated by differences in their distribution between two phases, one of which is stationary (stationary phase) while the other (the mobile phase) moves through it in a definite direction. The substances must interact with the stationary phase to be retained and separated by it.

Gas chromatography is a well known technique for fractionating and determining the relative amounts of various components in a sample containing a mixture of compounds of differing volatilities. In the conventional such process, the sample is vaporized and the entire resulting quantity of gases is passed through an analytical chromatography column. Chromatographic processes such as gas chromatography can rapidly determine the volatiles content of a multicomponent sample, such as would be produced by Gliocladium isolate C-13 (as described below). Processes for gas chromatography are described, e.g., in U.S. Pat. Nos. 4,780,284, 5,057,126, and 6,838,288, each of which is hereby incorporated by reference in its entirety.

In liquid absorption chromatography, the stationary phase consists of a tubular column packed with an absorbent material. The mobile phase for carrying an analysis sample through the column, commonly referred to as the carrier, is a solvent mixture comprising two or more miscible liquids, which are introduced into the column. An equilibrium is established for the individual components of a sample mixture according to the “attraction” of each to the stationary phase and according to the solubility of each component in the carrier solvent. The rate at which a solute passes through the column chromatograph is dependent upon the equilibria existing for the components, and separations of the components occur where the distributions differ.

Pressure Swing Adsorption (PSA) is a technology used to separate some gas species from a mixture of gases under pressure according to the species' molecular characteristics and affinity for an adsorbent material. It operates at near-ambient temperatures and so differs from cryogenic distillation techniques of gas separation. Special adsorptive materials (e.g., zeolites) are used as a molecular sieve, preferentially adsorbing the target gas species at high pressure. The process then swings to low pressure to desorb the adsorbent material.

The invention also provides for a kit comprising one or more containers filled with one or more of the ingredients of the compositions of the invention. The present invention provides kits that can be used in the above methods. In one embodiment, a kit comprises a Gliocladium spp., such isolate C-13, in one or more containers. The organism is supplied frozen in media, freeze dried and/or as spores. In one embodiment, the invention comprises a kit for making VOCs comprising Gliocladium spp. and instructions for growing said Gliocladium spp. under optimal conditions for optimal VOC production. The methods in the instructions may include specific bioreactor volumes, purification schemes, optimal temperature, pH, dissolved O₂, CO₂, and/or other conditions. The kit may also include blueprints for the design of a factory to produce VOCs by growing Gliocladium spp. in large bioreactor vessels. In another embodiment, the kit further comprises growth media. In another embodiment, said media is an oatmeal based media. The media contained in the containers of these kits may be present as 1× ready-to-use formulations, or as more concentrated solutions (for example 2×, 5×, 10×, 20×, 25×, 50×, 10×, 500×, 1000× or higher). In addition, the media can be supplied in dry powder. Thus, a kit can comprise a dry power of the medium of the invention and a liquid to suspend the media. The liquid may be water or buffers known in the art. Filters for sterilization of the media may also be provided. The kit may also comprise methods for growing said Gliocladium spp. under optimal conditions for optimal VOC (e.g. hydrocarbon) production.

As described herein, the VOCs (e.g. hydrocarbons) of the present invention are useful for the production of biofuels, jet fuels, plastics, plasticizers, antibiotics, rubber, fuel additives, and/or adhesives. As will be appreciated by one of skill in the art, hydrocarbons can also be used for electrical power generation and heating. The chemical, petrochemical, plastics and rubber industries are also dependent upon hydrocarbons as raw materials for their products. Moreover, most industrially significant synthetic chemicals are derived from hydrocarbons. See, for example, Hydrocarbon Chemistry, George A. Olah and Arpad Molnar, 2003 (Wiley-Interscience, 871 pages). The hydrocarbons produced by Gliocladium isolate C-13 can supply the materials for these industries. In some embodiments, hydrocarbons made by Gliocladium isolate C-13 can be used for biofuels such as biodiesel.

As used herein, the term “biofuel” refers generally to any fuel that derives from biomass, i.e. recently living organisms or their metabolic byproducts, such as manure from cows. A biofuel may be further defined as a fuel derived from a metabolic product of a living organism. It is a renewable energy source, unlike other natural resources such as petroleum, coal and nuclear.

U.S. Pat. No. 5,713,965 describes a process of producing biofuels by lipase-catalyzed transesterification of alcohols utilizing inexpensive feedstocks such as animal fats, vegetable oils, rendered fats and restaurant grease as substrates. U.S. Patent Application Publication No. 20040074760 provides a method for the production of biofuels including applying radio frequency (RF) or microwave energy (ME) to at least one of a plant oil, an animal oil and a mixture thereof to produce a biofuel. U.S. Patent Application Publication No. 20070033863 provides methods of producing biofuels, such as biodiesel, from trap grease.

As will be appreciated by one of skill in the art, microorganisms such Gliocladium isolate C-13 can be used in combination with one or more microbes (e.g. yeasts or other bacteria) for the large scale production of biofuels. For example, U.S. Patent Application Publication No. 20070178569 discloses that Clostridium phytofermentans, such as strain ISDg^{T}, can ferment a material that is or includes a carbohydrate, or a mixture of carbohydrates, into a combustible fuel, e.g., ethanol, propanol and/or hydrogen, on a large scale.

In certain embodiments, the VOCs can be used to produce biodiesel fuels. As used herein, the term “biodiesel fuel” refers generally to diesel-equivalent processed fuel derived from biological sources which can be used in unmodified diesel-engine vehicles. Biodiesels are attractive for fuels, and some other uses, because they have a low vapor pressure, are non-toxic and are stable, as per HMIS regulation, and do not deteriorate or detonate upon mild heating. Chemically, biodiesels are generally defined as the mono alkyl esters of long chain fatty acids derived from renewable lipid sources.

Biodiesel can be obtained from oleaginous seeds, in particular from rapeseed, sunflower and soy bean seeds. The seeds can be subjected to grinding and/or solvent extraction treatments (e.g., with n-hexane) in order to extract the oil, which is essentially constituted by triglycerides of saturated and unsaturated (mono- and poly-unsaturated, in mixture with each other, in proportions depending on the selected oleaginous seed), C₁₆-C₂₂ fatty acids. The oil can be filtered and refined in order to remove any possible free fats and phospholipids present, and submitted to a trans-esterification reaction with methanol in order to prepare the methyl esters of the fatty acids, which constitute biodiesel. See, for example, U.S. Pat. No. 5,891,203.

U.S. Pat. No. 6,887,283 provides for the transesterification of triglyceride-containing substances and esterification of free fatty acid-containing substances with alcohol to produce alkyl esters of triglycerides, a desirable additive or alternative for petroleum diesel fuel or lubricants.

U.S. Pat. No. 7,112,229 provides a process for producing biodiesel fuel using triglyceride-rich oleagineous seed directly in a transesterification reaction in the presence of an alkaline alkoxide catalyst.

In an exemplary embodiment, the VOC produced by Gliocladium isolate C-13 is used to produce jet fuel. The primary volatile compound produced by Gliocladium isolate C-13 is 1,3,5,7-cyclooctatetraene or [8]-annulene, which by itself, is an effective inhibitor of fungal growth and was the main material used by the Germans in jet fuel in World War II. Other hydrocarbons produced by this organism of general interest for bioenergy are 1-octene, 1,3 octadiene and 7-octen-4-ol. In additional examples, a blend useful as a fuel includes a reduced hydrogen compound produced from Gliocladium isolate C-13 (NRRL 50072). Such compounds can also be used for plasticizers, antibiotics, fuel additives, and/or adhesives.

As noted above, one of the primary hydrocarbons produced by Gliocladium isolate C-13 is 1-octene. The primary, even overwhelming, use of 1-octene is as a comonomer in production of polyethylene. Thus, in one embodiment, the 1-octene produced by Gliocladium isolate C-13 is used to produce polyethylene. High density polyethylene (HDPE) and linear low density polyethylene (LLDPE) use approximately 2-4% and 8-10% of comonomers, respectively. Another significant use of 1-octene is for production of linear aldehyde via OXO synthesis (hydroformylation) for later production of the short-chain fatty acid nonionic acid, a carboxylic acid, by oxidation of an intermediate aldehyde or linear alcohols for plasticizer application by hydrogenation of the aldehyde.

In another embodiment, the invention provides products resulting from the oxidation of any unsaturated hydrocarbons produced by Gliocladium isolate C-13. The oxidation of unsaturated hydrocarbons by atmospheric oxygen with the aid of heterogeneous or homogeneous catalysts is an industrially important process. Thus, for example, by the catalytic oxidation of propylene by air, acetone and acrylic acid are obtained as products which are employed in the synthesis of many products prepared on a large industrial scale. Nevertheless, the oxidation of unsaturated hydrocarbons by atmospheric oxygen as a rule leads to product mixtures. Thus, for example, in the oxidation of propylene by atmospheric oxygen, in addition to acetone and acrylic acid, other oxygen-containing products, for example acrolein, propionic acid, propionaldehyde, acetic acid, CO₂, acetaldehyde or methanol, are also obtained.

Interestingly, the Gliocladium sp. described herein has not been found in any other country where the inventor has gone bioprospecting. After looking in Peru, Bolivia, Ecuador, South Africa, Indonesia, New Guinea, Thailand, South China, Nepal and Madagascar this particular Gliocladium sp. was not isolated. In addition, fungal endophytes may not permeate the whole tree or the area surrounding the tree, thus the inventor believes that it would be very difficult to isolate the same strain again, even from the same area where isolate C-13 was discovered and isolated by the inventor. Thus, the Gliocladium isolate C-13 (NRRL 50072, Gliocladium roseum) may not be found without undue experimentation and a whole lot of luck.

In some aspects, the invention also comprises, an isolated strain of a Gliocladium, wherein Gliocladium isolate C-13 (deposited as NRRL 50072) was serially propagated. When strains are serially propagated, some of the characteristics of the strain may change. Such changes include deletion or suppression of metabolic pathways, an increase in certain metabolic pathways, changes to the chromosome, genes and/or operons (e.g. via mutations or changes in the regulatory factors that control the expression level of said genes or operons). In one embodiment, the said strain of Gliocladium, has changes in the metabolic characteristic and/or genetic make-up as compared to Gliocladium isolate C-13 (of which said strain of a Gliocladium is a derivative). In another embodiment, said changes to the metabolic characteristics increase and/or decrease the production of the specific compounds listed in Table 4, 7, 8, or 9. In another embodiment, said genetic make-up would increase and/or decrease the production of the specific compounds listed in Table 4, 7, 8, or 9. Methods for isolating mutant cells with a desired characteristic are well known in the art. See, for example, U.S. Pat. No. 5,348,872, which is herein incorporated by reference in its entirety.

### Deposit of Biological Material

The following biological material has been deposited under the terms of the Budapest Treaty with the Agricultural Research Service Patent Culture Collection, Northern Regional Research Center, 1815 University Street, Peoria, Ill., 61604, and given the following accession number:

| Deposit | Accession Number | Date of Deposit |
|---|---|---|
| Gliocladium sp. | NRRL 50072 | Oct. 9, 2007 |

The strain has been deposited under conditions that assure that access to the culture will be available during the pendency of this patent application to one determined by the Commissioner of Patents and Trademarks to be entitled thereto under 37 C.F.R. §1.14 and 35 U.S.C. §122. The deposit represents a substantially pure culture of the deposited strain. The deposit is available as required by foreign patent laws in countries wherein counterparts of the subject application, or its progeny are filed. However, it should be understood that the availability of a deposit does not constitute a license to practice the subject invention in derogation of patent rights granted by governmental action.

Based on this deposit, the entire genome of isolate C-13 (NRRL 50072) is hereby incorporated into and included in this filing.

Thus, the invention comprises identifying and cloning genes that encode for VOC production from the Gliocladium C-13 genome. In one embodiment, the Gliocladium genome is probed for the gene or genes (e.g. an operon) that encode the synthetic pathways that produce said VOC. Thus, the invention encompasses an isolated nucleic acid molecule from Gliocladium isolate C-13 (NRRL 50072) encoding a polypeptide involved in the synthesis or production of VOCs. In another embodiment, said VOC is a hydrocarbon. In another embodiment, said hydrocarbon is a compound selected from Table 4, 7, 8, or 9. In another embodiment, said hydrocarbon is selected from the group consisting of 1,3,5,7,-cyclooctatatraene, 1-octene, 1,3 octadiene, 7-octen-4-ol. In another embodiment, an isolated nucleic acid molecule is at least 65%, at least 70%, at least 75%, at least 80%, at least 85%, at least 90%, at least 95%, at least 96%, at least 97%, at least 98%, or at least 99% identical to said isolated nucleic acid molecule from Gliocladium isolate C-13 (NRRL 50072). In another embodiment, a polypeptide sequence is at least 65%, at least 70%, at least 75%, at least 80%, at least 85%, at least 90%, at least 95%, at least 96%, at least 97%, at least 98%, or at least 99% identical to a polypeptide from Gliocladium isolate C-13 (NRRL 50072)

Methods to clone and/or probe genomes for synthetic pathways are known in the art. These methods include creating cDNA and/or genomic libraries, screening the libraries for genes that produce the VOC synthetic pathways. Thus, the invention comprises a DNA and/or chromosomal library of Gliocladium isolate C-13 (NRRL 50072). In one embodiment, said library is cloned into a vector that can replicate in a prokaryotic cell and/or eukaryotic cell. In another embodiment, said eukaryotic cell is a fungal cell. In another embodiment, said library is a lambda phage, Yeast Artificial Chromosome (YAC, see U.S. Pat. Nos. 4,889,806 and 5,643,763, herein incorporated by reference in their entireties), Bacterial Artificial Chromosome (BAC, see 5,874,259, herein incorporated by reference in their entireties), and/or cDNA. In another embodiment, said library is screened for production of VOCs. In another embodiment, said VOC is a hydrocarbon. In another embodiment, said hydrocarbon is selected from the Table 4, 7, 8, or 9. In an exemplary embodiment, said hydrocarbon is selected from the group consisting of 1,3,5,7,-cyclooctatatraene, 1-octene, 1,3 octadiene, 7-octen-4-ol.

Another method for determining the gene, genes and/or operon(s) that encode for the production of VOCs include mutagenizing the genome of Gliocladium isolate C-13 (NRRL 50072) and looking for an increase, addition, reduction or removal of a specific VOC. This can be accomplished via chemical and/or transposon mutagenesis. Once a gene, genes and/or operon(s) is identified, said gene, genes or operon(s) can be cloned and/or isolated. Thus, one embodiment of the invention comprises an isolated nucleic acid of Gliocladium isolate C-13 (NRRL 50072) wherein said nucleic acid molecule is cloned into a vector. In another embodiment, said nucleic acid molecule encodes for a gene, genes, or operon(s) that encode for proteins involved in the production of VOCs. In another embodiment, said vector autonomously replicates or integrates into the host's chromosome. In another embodiment, said vector is transformed or transfected into a heterologous cell. In another embodiment, said heterologous cell is selected from the group consisting of a prokaryotic or eukaryotic cell.

The genetic manipulation and/or transformation of an organism, such as a Gliocladium species is well known to those skilled in the art. See, for example, Dave et al., Appl Microbiol Biotechnol, 1994, 41(3):352-358; Garcia-Granados et al., Phytochemistry, 2004, 65(1):107-115; Mikkelsen et al., FEMS Microbiol Lett., 2003, 223(1):135-139; Takahashi-Ando et al., Biochem J., 2002, 365(Pt 1):1-6; Ossanna et al., Appl Environ Microbiol, 1990, 56(10):3052-3056; Lorito et al., Curr Genet, 1993, 24(4):349-356; and Gan et al., J Microbiol, 2007, 45(5):422-430, each of which herein is incorporated by reference in their entireties. See, also, Trichoderma and Gliocladium Volume 1: Basic biology, taxonomy and genetics, C. P. Kubicek and Gary E. Harman (editors), July 1998 (ISBN-10: 0748405720; CRC, 1rst Edition, 300 pages) and Trichoderma and Gliocladium, C. P. Kubicek and Gary E. Harman (editors), June 1998 (ISBN-10: 0748408053; CRC, 1^{st} Edition, 300 pages), both of which are incorporated herein in their entireties.

The invention also encompasses variants and fragments of polynucleotides and/or proteins of said Gliocladium isolate C-13 that produce or are part of the pathway(s) that produce VOCs. The variants may contain alterations in the nucleotide and/or amino acid sequences of the constituent proteins. The term “variant” with respect to a polypeptide refers to an amino acid sequence that is altered by one or more amino acids with respect to a reference sequence. The variant can have “conservative” changes, or “nonconservative” changes, e.g., analogous minor variations can also include amino acid deletions or insertions, or both. In addition, the nucleotides can be sequenced to ensure that the correct coding regions were cloned and do not contain any unwanted mutations.

Functional fragments and variants of a polypeptide include those fragments and variants that maintain one or more functions of the parent polypeptide. It is recognized that the gene or cDNA encoding a polypeptide can be considerably mutated without materially altering one or more the polypeptide's functions. First, the genetic code is well-known to be degenerate, and thus different codons encode the same amino acids. Second, even where an amino acid substitution is introduced, the mutation can be conservative and have no material impact on the essential function(s) of a protein. See, e.g., Stryer Biochemistry 3^{rd} Ed., 1988. Third, part of a polypeptide chain can be deleted without impairing or eliminating all of its functions. Fourth, insertions or additions can be made in the polypeptide chain for example, adding epitope tags, without impairing or eliminating its functions (Ausubel et al. J. Immunol. 159(5): 2502-12, 1997). Other modifications that can be made without materially impairing one or more functions of a polypeptide include, for example, in vivo or in vitro chemical and biochemical modifications or the incorporation of unusual amino acids. Such modifications include, for example, acetylation, carboxylation, phosphorylation, glycosylation, ubiquination, labeling, e.g., with radionucleotides, and various enzymatic modifications, as will be readily appreciated by those well skilled in the art. A variety of methods for labeling polypeptides, and labels useful for such purposes, are well known in the art, and include radioactive isotopes such as 32^{P}, ligands which bind to or are bound by labeled specific binding partners (e.g., antibodies), fluorophores, chemiluminescent agents, enzymes, and anti-ligands. Functional fragments and variants can be of varying length. For example, some fragments have at least 10, 25, 50, 75, 100, 200, or even more amino acid residues.

These mutations can be natural or purposely changed. In some embodiments, mutations containing alterations that produce silent substitutions, additions, or deletions, but do not alter the properties or activities of the encoded protein or how the proteins are made are an embodiment of the invention. Nucleotide variants can be produced for a variety of reasons, e.g., to optimize codon expression for a particular host. In other embodiments, such changes may reduce or increase the desired result (e.g. increase or decrease production of VOCs or remove a splice site). The nucleotides can be subcloned into an expression vector for expression in any cell.

To mutate an organism is the process of causing a change in the sequence of a genetic material (usually DNA or RNA) of a cell or organism (such as a bacterium). Mutations can be intentionally introduced into genetic material using molecular techniques well known in the art (e.g., site-directed mutagenesis, PCR mutagenesis and others). As used herein, the term “mutating” may be used to refer to restoring a variant (or previously mutated) polynucleotide (also referred to as a SNP) to its wild-type sequence.

The expression vectors of the present invention will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Among vectors preferred are virus vectors, such as baculovirus, poxvirus (e.g., vaccinia virus, avipox virus, canarypox virus, fowlpox virus, raccoonpox virus, swinepox virus, etc.), adenovirus (e.g., canine adenovirus), herpesvirus, and retrovirus. Other vectors that can be used with the invention comprise vectors for use in bacteria, which comprise pQE70, pQE60 and pQE-9, pBluescript vectors, Phagescript vectors, pNH8A, pNH16a, pNH18A, pNH46A, ptrc99a, pKK223-3, pKK233-3, pDR540, pRIT5. Among preferred eukaryotic vectors are pFastBac1 pWINEO, pSV2CAT, pOG44, pXT1 and pSG, pSVK3, pBPV, pMSG, and pSVL. Other suitable vectors will be readily apparent to the skilled artisan.

In one embodiment, an isolated polynucleotide from Gliocladium isolate C-13 (NRRL 50072) is inserted into a synthetic genome. In another embodiment, said synthetic genome is transformed, transfected or infected into a prokaryotic or eukaryotic cell creating a new organism. In another embodiment, said new organism is cultured in such a way to produce the desired product that was added to the synthetic genome. Such product comprises at least one VOC (e.g. hydrocarbon) derived from a gene, genes and/or operon(s) of Gliocladium isolate C-13 (NRRL 50072).

A synthetic genome is a genome originating, at least in part, from the extracellular chemical synthesis of a sequenced of nucleotide bases. This permits a person of skill in the art to manipulate specific genes or create new genomes by piecing synthetic genes from known organisms. Synthetic genomes also comprise genomes that have the minimal amount of genes for an organism to live. These genomes will be substantially smaller than a wild type genome (see Kobayashi et al., (2003) PNAS 100: 4678 to 4683). These genomes can then be used to produce a desired product once the gene, genes, and/or operon(s) are inserted into the synthetic genome and said genome is inserted into an organism. The added gene, genes, and/or operons add the desired biological capabilities that allow metabolic activity and replication of the newly created organism.

Methods are also provided herein for sequencing the whole genome for Gliocladium isolate C-13 and for isolating and cloning nucleic acids from such isolate. For example, methods for the molecular cloning of Gliocladium isolate C-13 biosynthetic genes, the characterization of the individual genes (such as those involved in production, or regulation of production, of a hydrocarbon), the proteins encoded thereby, and modification of gene(s) (or protein(s)) to produce desired products, such as hydrocarbons, are disclosed herein.

The current disclosure provides Gliocladium isolate C-13 (NRRL 50072). Methods of sequencing whole genomes of organisms are well known to those of skill in the art. Therefore, undue experimentation would not be required for one of skill in the art to sequence the genome of the presently disclosed isolate C-13. In one example, a whole genome random sequencing method (Fleischmann et al., Science 269:496, 1995; Fraser, et al., Science 270:397, 1995) can be used to obtain the complete genome sequence for isolate C-13. In one example, a Gliocladium isolate C-13 biosynthetic gene or set of genes involved, for instance, in production of a hydrocarbon, is cloned. Gliocladium isolate C-13 biosynthetic gene(s) can be isolated using techniques well known in the art, including a strategy based on Gliocladium predicted enzymatic proteins. In a particular example, a chromosome of the native hydrocarbon producer, Gliocladium isolate C-13, or suitable surrogate host cells harboring a Gliocladium isolate C-13 gene (or set of two or more thereof), can be modified through deletion, replacement or disruption of segments of the host chromosome to result in the generation of hydrocarbons, such as those listed in Tables 4, 7, 8, and 9. Alternatively, specific genes involved in hydrocarbon production can be deleted or disrupted (or duplicated, or genetically modified to produce a different variant protein) to alter the processing or synthesis of certain hydrocarbon compounds, or to rebalance or alter the composition of hydrocarbons produced. For example, specific genes from Gliocladium isolate C-13 can be deleted or disrupted to increase production of hydrocarbons such as those discussed herein.

With respect to Gliocladium isolate C-13 (and particularly with its deposit as NRRL 50072), in vitro nucleic acid amplification (such as PCR) may be utilized as a simple method for producing nucleic acid sequences encoding one or more of the desired biosynthetic enzymes involved in the production of hydrocarbons.

Amplification of a nucleic acid molecule (such as a DNA or RNA molecule) refers to use of a technique that increases the number of copies of a nucleic acid molecule in a sample. An example of amplification is the polymerase chain reaction (PCR), in which a sample is contacted with a pair of oligonucleotide primers under conditions that allow for the hybridization of the primers to a nucleic acid template in the sample. The primers are extended under suitable conditions, dissociated from the template, re-annealed, extended, and dissociated to amplify the number of copies of the nucleic acid. The product of amplification can be characterized by such techniques as electrophoresis, restriction endonuclease cleavage patterns, oligonucleotide hybridization or ligation, and/or nucleic acid sequencing.

Other examples of amplification methods include strand displacement amplification, as disclosed in U.S. Pat. No. 5,744,311; transcription-free isothermal amplification, as disclosed in U.S. Pat. No. 6,033,881; repair chain reaction amplification, as disclosed in WO 90/01069; ligase chain reaction amplification, as disclosed in EP-A-320,308; gap filling ligase chain reaction amplification, as disclosed in U.S. Pat. No. 5,427,930; and NASBA™ RNA transcription-free amplification, as disclosed in U.S. Pat. No. 6,025,134. An amplification method can be modified, including for example by additional steps or coupling the amplification with another protocol.

To obtain the necessary template nucleic acid for PCR and/or RT-PCR, DNA and/or RNA can be extracted from cells by any one of a variety of methods well known to those of ordinary skill in the art. Sambrook et al. (In Molecular Cloning: A Laboratory Manual, Cold Spring Harbor Laboratory Press, New York, 1989) and Ausubel et al. (In Current Protocols in Molecular Biology, Greene Publ. Assoc. and Wiley-Intersciences, 1992) provide representative descriptions of methods for RNA or DNA isolation.

In some examples, RNA or DNA may be extracted from Gliocladium isolate C-13 cells. Extracted RNA is used, for example, as a template for performing reverse transcription (RT)-PCR amplification to produce cDNA. Representative methods and conditions for RT-PCR are described by Kawasaki et al. (In PCR Protocols, A Guide to Methods and Applications, Innis et al. (eds.) 21-27 Academic Press, Inc., San Diego, Calif., 1990).

The selection of amplification primers will be made according to the portion(s) of the DNA that is to be amplified. In one embodiment, primers may be chosen to amplify a segment of a DNA (e.g., a specific ORF or set of adjacent ORFs) or, in another embodiment, the entire DNA molecule. As used herein, the term “open reading frame” or “ORF” refers to a series of nucleotide triplets (codons) coding for amino acids without any internal termination codons. These sequences are usually translatable into a peptide/polypeptide/protein/polyprotein.

Variations in amplification conditions may be required to accommodate primers and amplicons of differing lengths and composition; such considerations are well known in the art and are discussed for instance in Innis et al. (PCR Protocols, A Guide to Methods and Applications, Academic Press, Inc., San Diego, Calif., 1990). It will be appreciated that many different primers may be derived from the provided nucleic acid sequences.

Primers are short nucleic acid molecules, for instance DNA oligonucleotides, usually 7 nucleotides or more in length, for example that hybridize to contiguous complementary nucleotides or a sequence to be amplified. Longer DNA oligonucleotides may be about 15, 20, 25, 30 or 50 nucleotides or more in length. Primers can be annealed to a complementary target DNA strand by nucleic acid hybridization to form a hybrid between the primer and the target DNA strand, and then the primer extended along the target DNA strand by a DNA polymerase enzyme. Primer pairs can be used for amplification of a nucleic acid sequence, for example, by the PCR or other nucleic-acid amplification methods known in the art, as described above.

A probe comprises an identifiable, isolated nucleic acid that recognizes a target nucleic acid sequence. A probe includes a nucleic acid that is attached to an addressable location, a detectable label or other reporter molecule and that hybridizes to a target sequence. Typical labels include radioactive isotopes, enzyme substrates, co-factors, ligands, chemiluminescent or fluorescent agents, haptens, and enzymes. Methods for labeling and guidance in the choice of labels appropriate for various purposes are discussed, for example, in Sambrook et al. (ed.), Molecular Cloning: A Laboratory Manual, 2^{nd} ed., vol. 1-3, Cold Spring Harbor Laboratory Press, Cold Spring Harbor, N.Y., 1989 and Ausubel et al. Short Protocols in Molecular Biology, 4^{th} ed., John Wiley & Sons, Inc., 1999.

Methods for preparing and using nucleic acid probes and primers are described, for example, in Sambrook et al. (ed.), Molecular Cloning: A Laboratory Manual, 2^{nd} ed., vol. 1-3, Cold Spring Harbor Laboratory Press, Cold Spring Harbor, N.Y., 1989; Ausubel et al. Short Protocols in Molecular Biology, 4^{th} ed., John Wiley & Sons, Inc., 1999; and Innis et al. PCR Protocols, A Guide to Methods and Applications, Academic Press, Inc., San Diego, Calif., 1990. Amplification primer pairs can be derived from a known sequence, for example, by using computer programs intended for that purpose such as PRIMER (Version 0.5, 1991, Whitehead Institute for Biomedical Research, Cambridge, Mass.). One of ordinary skill in the art will appreciate that the specificity of a particular probe or primer increases with its length. Thus, in order to obtain greater specificity, probes and primers can be selected that comprise at least 20, 25, 30, 35, 40, 45, 50 or more consecutive nucleotides of a target nucleotide sequences.

Re-sequencing of amplification products obtained by any amplification procedure is recommended to facilitate confirmation of the amplified sequence, and to provide information on natural variation between a Gliocladium isolate C-13 and the amplified sequence. Oligonucleotides derived from any of the Gliocladium isolate C-13 sequences may be used in sequencing, for instance, the corresponding Gliocladium isolate C-13 (or Gliocladium isolate C-13-related) amplicon.

As used herein, an “oligonucleotide” refers generally to a nucleic acid molecule usually comprising a length of 300 bases or fewer. The term often refers to single-stranded deoxyribonucleotides, but it can refer as well to single- or double-stranded ribonucleotides, RNA:DNA hybrids and double-stranded DNAs, among others. The term “oligonucleotide” also includes oligonucleosides (that is, an oligonucleotide minus the phosphate) and any other organic base polymer. In some examples, oligonucleotides are about 10 to about 90 bases in length, for example, 12, 13, 14, 15, 16, 17, 18, 19 or 20 bases in length. Other oligonucleotides are about 25, about 30, about 35, about 40, about 45, about 50, about 55, about 60 bases, about 65 bases, about 70 bases, about 75 bases or about 80 bases in length. Oligonucleotides may be single-stranded, for example, for use as probes or primers, or may be double-stranded, for example, for use in the construction of a mutant gene. Oligonucleotides can be either sense or anti-sense oligonucleotides. An oligonucleotide can be modified as discussed above in reference to nucleic acid molecules. Oligonucleotides can be obtained from existing nucleic acid sources (for example, genomic or cDNA), but can also be synthetic (for example, produced by laboratory or in vitro oligonucleotide synthesis)

Both conventional hybridization and PCR amplification procedures may be utilized to clone sequences encoding orthologs of Gliocladium isolate C-13 genes (e.g., synthetic genes, such as those producing proteins involved in hydrocarbon production), or Gliocladium isolate C-13 ORFs. Common to both of these techniques is the hybridization of probes or primers that are derived from the Gliocladium isolate C-13 gene or set of genes with or without the upstream and downstream flanking regions or Gliocladium isolate C-13 ORFs nucleic acid sequences. Furthermore, the hybridization may occur in the context of Northern blots, Southern blots, or PCR.

Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between to distinct nucleic acid sequences or two distinct regions of the same nucleic acid sequence.

“Specifically hybridizable” and “specifically complementary” are terms that indicate a sufficient degree of complementarity such that stable and specific binding occurs between the oligonucleotide (or its analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired, for example under physiological conditions in the case of in vivo assays or systems. Such binding is referred to as specific hybridization.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ and/or Mg⁺⁺ concentration) of the hybridization buffer will determine the stringency of hybridization, though wash times also influence stringency. Calculations regarding hybridization conditions required for attaining particular degrees of stringency are discussed by Sambrook et al. (ed.), Molecular Cloning: A Laboratory Manual, 2^{nd} ed., vol. 1-3, Cold Spring Harbor Laboratory Press, Cold Spring Harbor, N.Y., 1989, chapters 9 and 11; and Ausubel et al. Short Protocols in Molecular Biology, 4^{th} ed., John Wiley & Sons, Inc., 1999.

For purposes of the present disclosure, “stringent conditions” encompass conditions under which hybridization will only occur if there is less than 25% mismatch between the hybridization molecule and the target sequence. “Stringent conditions” may be broken down into particular levels of stringency for more precise definition. Thus, as used herein, “moderate stringency” conditions are those under which molecules with more than 25% sequence mismatch will not hybridize; conditions of “medium stringency” are those under which molecules with more than 15% mismatch will not hybridize, and conditions of “high stringency” are those under which sequences with more than 10% mismatch will not hybridize. Conditions of “very high stringency” are those under which sequences with more than 6% mismatch will not hybridize.

Direct PCR amplification may be performed on DNA libraries prepared from Gliocladium isolate C-13, or RT-PCR may be performed using RNA extracted from Gliocladium isolate C-13 cells using standard methods. PCR primers will generally comprise at least 10 consecutive nucleotides of a Gliocladium isolate C-13 gene with or without the upstream and downstream flanking regions or Gliocladium isolate C-13 ORF nucleic acid sequences. One of skill in the art will appreciate that sequence differences between a Gliocladium isolate C-13 gene or Gliocladium isolate C-13 ORF nucleic acid sequence and the target nucleic acid to be amplified may result in lower amplification efficiencies. To compensate for this, longer PCR primers or lower annealing temperatures may be used during the amplification cycle. Whenever lower annealing temperatures are used, sequential rounds of amplification using nested primer pairs may be useful to enhance amplification specificity.

Orthologs of the disclosed Gliocladium isolate C-13 biosynthetic proteins are likely present in a number of other members of the Gliocladium genus and in other isolates of Gliocladium spp. from Eucryphia cordifolia. With the deposit of the Gliocladium isolate C-13, the nucleic acid sequence of the disclosed Gliocladium isolate C-13 and its ORFs, the cloning by standard methods of protein-encoding DNA (such as, ORFs) and genes that encode Gliocladium sp. synthetic enzyme orthologs in these other organisms is now enabled. Orthologs of the disclosed Gliocladium sp. biosynthetic enzymes and proteins have a biological activity or function as disclosed herein, including for example the ability to produce hydrocarbons or a precursor to a specific hydrocarbon.

Orthologs will generally share at least 65% sequence identity with the nucleic acid sequences encoding the disclosed Gliocladium isolate C-13 biosynthetic proteins. In specific embodiments, orthologous Gliocladium sp. gene(s) or Gliocladium sp. ORFs may share at least 70%, at least 75%, at least 80% at least 85%, at least 90%, at least 91%, at least 93%, at least 95%, or at least 98% sequence identity with Gliocladium isolate C-13 nucleotide or amino acid sequences in the deposit strain NRRL 50072, as applicable.

For conventional hybridization techniques, the hybridization probe is preferably conjugated with a detectable label such as a radioactive label, and the probe is preferably at least 10 nucleotides in length. As is well known in the art, increasing the length of hybridization probes tends to give enhanced specificity. A labeled probe derived from a Gliocladium sp. gene set or Gliocladium sp. ORFs nucleic acid sequence may be hybridized to a bacterial DNA library and the hybridization signal detected using methods known in the art. The hybridizing colony or plaque (depending on the type of library used) is purified and the cloned sequence contained in that colony or plaque isolated and characterized.

In specific examples, genomic library construction can be accomplished rapidly using a variety of cosmid or fosmid systems that are commercially available (Stratagene, Epicentre). Advantageously, these systems minimize instability of the cloned DNA. In such examples, genomic library screening is followed by cosmid or fosmid isolation, grouping into families of overlapping clones and analysis to establish sequence identity. Cosmid end sequencing can be used to obtain preliminary information regarding the relevance of a particular clone based on expected pathway characteristics predicted from the natural product structure and its presumed biosynthetic origin.

Orthologs of a Gliocladium sp. gene or set of genes (+/−upstream or downstream flanking regions) or Gliocladium sp. ORFs nucleic acid sequences alternatively may be obtained by immunoscreening of an expression library. Further, the corresponding proteins can be expressed and purified in a heterologous expression system (e.g., E. coli) and used to raise antibodies (monoclonal or polyclonal) specific for the Gliocladium sp. biosynthetic enzymes or proteins, such as those involved in the production of hydrocarbons, including hydrocarbons provided in Tables 4, 7, 8, or 9. Antibodies also may be raised against synthetic peptides derived from the Gliocladium sp. biosynthetic enzymes or proteins. Methods of raising antibodies are well known in the art and are described generally in Harlow and Lane, Antibodies, A Laboratory Manual, Cold Springs Harbor, 1988. Such antibodies can be used to screen an expression library produced from bacteria. For example, this screening will identify the Gliocladium isolate C-13 orthologs. The selected DNAs can be confirmed by sequencing and enzyme activity assays.

Oligonucleotides derived from Gliocladium isolate C-13 nucleic acid sequences encoding ORFs, or fragments of these nucleic acid sequences, are encompassed within the scope of the present disclosure. Such oligonucleotides may be used, for example, as probes or primers. In one embodiment, oligonucleotides may comprise a sequence of at least 10 consecutive nucleotides of a Gliocladium sp. gene (+/−upstream and downstream flanking regions) or a Gliocladium sp. ORF nucleic acid sequence. If these oligonucleotides are used with an in vitro amplification procedure (such as PCR), lengthening the oligonucleotides may enhance amplification specificity. Thus, in other embodiments, oligonucleotide primers comprising at least 15, 20, 25, 30, 35, 40, 45, 50, or more consecutive nucleotides of these sequences may be used. In another example, a primer comprising 30 consecutive nucleotides of a nucleic acid molecule encoding a Gliocladium sp. biosynthetic enzyme will anneal to a target sequence, such as a Gliocladium sp. gene (+/−upstream and downstream flanking regions) or an Gliocladium isolate C-13 homolog present in a DNA library from another Gliocladium sp. species (or other Gliocladium isolate C-13-producing species), with a higher specificity than a corresponding primer of only 15 nucleotides. In order to obtain greater specificity, probes and primers can be selected that comprise at least 17, 20, 23, 25, 30, 35, 40, 45, 50 or more consecutive nucleotides of Gliocladium isolate C-13 gene (+/−upstream and downstream flanking regions) or a Gliocladium isolate C-13 ORF nucleotide sequences. In particular examples, probes or primers can be at least 100, 250, 500, 600 or 1000 consecutive nucleic acids of a disclosed Gliocladium isolate C-13 gene (+/−upstream and downstream flanking regions) or a Gliocladium isolate C-13 ORF sequence.

With the provision herein of the Gliocladium isolate C-13 biosynthetic proteins and corresponding nucleic acid sequences, the creation of variants of these sequences is now enabled. Variant Gliocladium isolate C-13 biosynthetic enzymes include proteins that differ in amino acid sequence from the disclosed prototype enzymes and still retain the biological activity/function of the prototype proteins, such as the ability to produce hydrocarbons, including production of one or more hydrocarbons listed in Table 4, 7, 8, or 9. Variant enzymes may also be stripped of particular activity/function.

In one embodiment, variant Gliocladium isolate C-13 biosynthetic proteins include proteins that differ in amino acid sequence from the disclosed Gliocladium isolate C-13 biosynthetic protein sequences but that share at least 65% amino acid sequence identity with such enzyme sequences. In other embodiments, other variants will share at least 70%, at least 75%, at least 80%, at least 85%, at least 90%, at least 95%, or at least 98% amino acid sequence identity. Manipulation of the disclosed Gliocladium isolate C-13 gene (+/−upstream and downstream flanking regions) and Gliocladium isolate C-13 ORF nucleotide sequences using standard procedures (e.g., site-directed mutagenesis or PCR), can be used to produce such variants. The simplest modifications involve the substitution of one or more amino acids for amino acids having similar biochemical properties. These so-called conservative substitutions are likely to have minimal impact on the activity of the resultant protein.

In some embodiments, the function of an Gliocladium isolate C-13 biosynthetic protein variant can be maintained if amino acid substitutions are introduced in regions outside of the conserved domains of the protein, where amino acid substitutions are less likely to affect protein function. As used herein, the term “domain” generally refers to a portion of a protein or nucleic acid that is structurally and/or functionally distinct from another portion of the protein or nucleic acid.

In another embodiment, more substantial changes in the Gliocladium isolate C-13 biosynthetic enzyme function or other protein features may be obtained by selecting amino acid substitutions that are less conservative than conservative substitutions. In one specific, non-limiting, embodiment, such changes include changing residues that differ more significantly in their effect on maintaining polypeptide backbone structure (e.g., sheet or helical conformation) near the substitution, charge or hydrophobicity of the molecule at the target site, or bulk of a specific side chain. The following specific, non-limiting, examples are generally expected to produce the greatest changes in protein properties: (a) a hydrophilic residue (e.g., seryl or threonyl) is substituted for (or by) a hydrophobic residue (e.g., leucyl, isoleucyl, phenylalanyl, valyl or alanyl); (b) a cysteine or proline is substituted for (or by) any other residue; (c) a residue having an electropositive side chain (e.g., lysyl, arginyl, or histidyl) is substituted for (or by) an electronegative residue (e.g., glutamyl or aspartyl); or (d) a residue having a bulky side chain (e.g., phenylalanine) is substituted for (or by) one lacking a side chain (e.g., glycine).

Variant Gliocladium isolate C-13 biosynthetic enzyme- or protein-encoding sequences may be produced by standard DNA mutagenesis techniques. In one specific, non-limiting, embodiment, M13 primer mutagenesis is performed. Details of these techniques are provided in Sambrook et al. (In Molecular Cloning: A Laboratory Manual, Cold Spring Harbor Laboratory Press, New York, 1989), Ch. 15. By the use of such techniques, variants may be created that differ from the disclosed Gliocladium isolate C-13 biosynthetic enzyme or protein sequences. DNA molecules and nucleotide sequences that are derivatives of those specifically disclosed herein, and which differ from those disclosed by the deletion, addition, or substitution of nucleotides while still encoding a protein having the biological activity of the prototype enzyme.

The terms “polypeptide” and “protein” as used herein are intended to encompass any amino acid sequence and include modified sequences such as glycoproteins. The term “polypeptide” is specifically intended to cover naturally occurring proteins, as well as those which are recombinantly or synthetically produced. The term recombinant refers generally to nucleic acids that have been altered by addition, substitution, or deletion of a portion of the nucleic acid.

Conservative amino acid substitutions are those substitutions that, when made, least interfere with the properties of the original protein, that is, the structure and especially the function of the protein is conserved and not significantly changed by such substitutions. Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain. Further information about conservative substitutions can be found, for instance, in Ben Bassat et al. (J. Bacteriol., 169:751-757, 1987), O'Regan et al. (Gene, 77:237-251, 1989), Sahin-Toth et al. (Protein Sci., 3:240-247, 1994), Hochuli et al. (Bio/Technology, 6:1321-1325, 1988) and in widely used textbooks of genetics and molecular biology.

The Blosum matrices are commonly used for determining the relatedness of polypeptide sequences. The Blosum matrices were created using a large database of trusted alignments (the BLOCKS database), in which pairwise sequence alignments related by less than some threshold percentage identity were counted (Henikoff et al., Proc. Natl. Acad. Sci. USA, 89:10915-10919, 1992). A threshold of 90% identity was used for the highly conserved target frequencies of the BLOSUM90 matrix. A threshold of 65% identity was used for the BLOSUM65 matrix. Scores of zero and above in the Blosum matrices are considered “conservative substitutions” at the percentage identity selected. The following table shows exemplary conservative amino acid substitutions.

TABLE 1

| | Very Highly- | Highly | |
|---|---|---|---|
| Original | Conserved | Conserved Substitutions | Conserved Substitutions |
| Residue | Substitutions | (from the Blosum90 Matrix) | (from the Blosum65 Matrix) |
| Ala | Ser | Gly, Ser, Thr | Cys, Gly, Ser, Thr, Val |
| Arg | Lys | Gln, His, Lys | Asn, Gln, Glu, His, Lys |
| Asn | Gln; His | Asp, Gln, His, Lys, Ser, Thr | Arg, Asp, Gln, Glu, His, Lys, Ser, Thr |
| Asp | Glu | Asn, Glu | Asn, Gln, Glu, Ser |
| Cys | Ser | None | Ala |
| Gln | Asn | Arg, Asn, Glu, His, Lys, Met | Arg, Asn, Asp, Glu, His, Lys, Met, Ser |
| Glu | Asp | Asp, Gln, Lys | Arg, Asn, Asp, Gln, His, Lys, Ser |
| Gly | Pro | Ala | Ala, Ser |
| His | Asn; Gln | Arg, Asn, Gln, Tyr | Arg, Asn, Gln, Glu, Tyr |
| Ile | Leu; Val | Leu, Met, Val | Leu, Met, Phe, Val |
| Leu | Ile; Val | Ile, Met, Phe, Val | Ile, Met, Phe, Val |
| Lys | Arg; Gln; Glu | Arg, Asn, Gln, Glu | Arg, Asn, Gln, Glu, Ser, |
| Met | Leu; Ile | Gln, Ile, Leu, Val | Gln, Ile, Leu, Phe, Val |
| Phe | Met; Leu; Tyr | Leu, Trp, Tyr | Ile, Leu, Met, Trp, Tyr |
| Ser | Thr | Ala, Asn, Thr | Ala, Asn, Asp, Gln, Glu, Gly, Lys, Thr |
| Thr | Ser | Ala, Asn, Ser | Ala, Asn, Ser, Val |
| Trp | Tyr | Phe, Tyr | Phe, Tyr |
| Tyr | Trp; Phe | His, Phe, Trp | His, Phe, Trp |
| Val | Ile; Leu | Ile, Leu, Met | Ala, Ile, Leu, Met, Thr |

In some examples, variants can have no more than 3, 5, 10, 15, 20, 25, 30, 40, 50, or 100 conservative amino acid changes (such as very highly conserved or highly conserved amino acid substitutions). In other examples, one or several hydrophobic residues (such as Leu, Ile, Val, Met, Phe, or Trp) in a variant sequence can be replaced with a different hydrophobic residue (such as Leu, Ile, Val, Met, Phe, or Trp) to create a variant functionally similar to the disclosed isolate C-13.

In some embodiments, the disruption of portions of a gene or set of genes may be achieved by use of the transposon-based method disclosed herein, which randomly inserts mutations into the gene or set of genes. These random mutations may be screened by restriction analysis in conjunction with DNA sequencing to select variants of the gene or set of genes with mutations in the sequences for specific enzymes involved in hydrocarbon production. Variants expressing one or more mutations may be selected. For example, mutations in portions of the gene or set of genes encoding for enzymes involved in the production of hydrocarbons may be selected from the random mutations.

In one embodiment, variants may differ from the disclosed sequences by alteration of the coding region to fit the codon usage bias of the particular organism into which the molecule is to be introduced. In other embodiments, the coding region may be altered by taking advantage of the degeneracy of the genetic code to alter the coding sequence such that, while the nucleotide sequence is substantially altered, it nevertheless encodes a protein having an amino acid sequence substantially similar to the disclosed Gliocladium isolate C-13 biosynthetic enzyme and protein amino acid sequences. For example, because of the degeneracy of the genetic code, four nucleotide codon triplets—(GCT, GCG, GCC and GCA)—code for alanine. The coding sequence of any specific alanine residue within a Gliocladium isolate C-13 biosynthetic enzyme, therefore, could be changed to any of these alternative codons without affecting the amino acid composition or characteristics of the encoded protein. Based upon the degeneracy of the genetic code, variant DNA molecules may be derived from the nucleic acid sequences disclosed herein using standard DNA mutagenesis techniques, as described above, or by synthesis of DNA sequences. Thus, this disclosure also encompasses nucleic acid sequences that encode a Gliocladium isolate C-13 biosynthetic enzyme or protein, but which vary from the disclosed nucleic acid sequences by virtue of the degeneracy of the genetic code.

A polynucleotide is said to “encode” a polypeptide if, in its native state or when manipulated by methods well known to those skilled in the art, it can be transcribed and/or translated to produce the mRNA for and/or the polypeptide or a fragment thereof. The anti-sense strand is the complement of such a nucleic acid, and the encoding sequence can be deduced therefrom. Unless otherwise specified, any reference to a DNA molecule is intended to include the reverse complement of that DNA molecule. Except where single-strandedness is required by the text herein, DNA molecules, though written to depict only a single strand, encompass both strands of a double-stranded DNA molecule. Thus, a reference to the nucleic acid molecule that encodes a specific protein, or a fragment thereof, encompasses both the sense strand and its reverse complement. Thus, for instance, it is appropriate to generate probes or primers from the reverse complement sequence of the disclosed nucleic acid molecules.

In one embodiment, variants of a Gliocladium isolate C-13 biosynthetic enzyme or protein may also be defined in terms of its sequence identity with the prototype Gliocladium isolate C-13 biosynthetic enzymes or variants. The phrase “sequence identity” generally refers to the similarity between two nucleic acid sequences, or two amino acid sequences. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith and Waterman (Adv. Appl. Math., 2:482, 1981); Needleman and Wunsch (J. Mol. Biol., 48:443, 1970); Pearson and Lipman (Proc. Natl. Acad. Sci., 85:2444, 1988); Higgins and Sharp (Gene, 73:237-44, 1988); Higgins and Sharp (CABIOS, 5:151-53, 1989); Corpet et al. (Nuc. Acids Res., 16:10881-90, 1988); Huang et al. (Comp. Appls Biosci., 8:155-65, 1992); and Pearson et al. (Meth. Mol. Biol., 24:307-31, 1994). Altschul et al. (Nature Genet., 6:119-29, 1994) presents a detailed consideration of sequence alignment methods and homology calculations.

The alignment tools ALIGN (Myers and Miller, CABIOS 4:11-17, 1989) or LFASTA (Pearson and Lipman, 1988) may be used to perform sequence comparisons (W. R. Pearson and the University of Virginia, “fasta20u63” version 2.0u63, release date December 1996). ALIGN compares entire sequences against one another, while LFASTA compares regions of local similarity. These alignment tools and their respective tutorials are available on the Internet at the NCSA website. Alternatively, for comparisons of amino acid sequences of greater than about 30 amino acids, the “Blast 2 sequences” function can be employed using the default BLOSUM62 matrix set to default parameters, (gap existence cost of 11, and a per residue gap cost of 1). When aligning short peptides (fewer than around 30 amino acids), the alignment should be performed using the “Blast 2 sequences” function, employing the PAM30 matrix set to default parameters (open gap 9, extension gap 1 penalties). The BLAST sequence comparison system is available, for instance, from the NCBI web site; see also Altschul et al., J. Mol. Biol., 215:403-10, 1990; Gish & States, Nature Genet., 3:266-72, 1993; Madden et al., Meth. Enzymol., 266:131-41, 1996; Altschul et al., Nucleic Acids Res., 25:3389-402, 1997; and Zhang and Madden, Genome Res., 7:649-56, 1997.

Orthologs (i.e. equivalent to proteins of other species) of proteins are in some instances characterized by possession of greater than 75% sequence identity counted over the full-length alignment with the amino acid sequence of specific protein using ALIGN set to default parameters. Proteins with even greater similarity to a reference sequence will show increasing percentage identities when assessed by this method, such as at least 80%, at least 85%, at least 90%, at least 92%, at least 95%, or at least 98% sequence identity. In addition, sequence identity can be compared over the full length of one or both binding domains of the disclosed fusion proteins.

When significantly less than the entire sequence is being compared for sequence identity, homologous sequences will typically possess at least 80% sequence identity over short windows of 10-20 nucleic acids or amino acids, and may possess sequence identities of at least 85%, at least 90%, at least 95%, or at least 99% depending on their similarity to the reference sequence. Sequence identity over such short windows can be determined using LFASTA; methods are described at the NCSA website. One of skill in the art will appreciate that these sequence identity ranges are provided for guidance only; it is entirely possible that strongly significant homologs could be obtained that fall outside of the ranges provided. Similar homology concepts apply for nucleic acids as are described for protein.

An alternative indication that two nucleic acid molecules are closely related is that the two molecules hybridize to each other under stringent conditions. Representative hybridization conditions are discussed above.

Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequences that each encode substantially the same protein.

Nucleic acid sequences that encode such proteins/fragments readily may be determined simply by applying the genetic code to the amino acid sequence of a Gliocladium isolate C-13 biosynthetic enzyme, protein or fragment thereof, and such nucleic acid molecules may readily be produced by assembling oligonucleotides corresponding to portions of the sequence.

Nucleic acid molecules that are derived from a Gliocladium isolate C-13 gene (+/−upstream and downstream flanking regions) and Gliocladium isolate C-13 ORF nucleic acid sequences include molecules that hybridize under low stringency, high stringency, or very high stringency conditions to the disclosed prototypical Gliocladium isolate C-13 gene or set of genes (+/−upstream and downstream flanking regions) and Gliocladium isolate C-13 ORFs and fragments thereof.

Nucleic acid molecules encoding one or more Gliocladium isolate C-13 biosynthetic enzyme or protein, and orthologs and homologs of these sequences, may be incorporated into transformation or expression vectors. As used herein, the term “vector” refers generally to a nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art. A transformed cell is a cell into which has been introduced a nucleic acid molecule by molecular biology techniques. The term encompasses all techniques by which a nucleic acid molecule might be introduced into such a cell, including transfection with viral vectors, transformation with plasmid vectors, and introduction of naked DNA by electroporation, lipofection, and particle gun acceleration.

Once Gliocladium isolate C-13 gene(s) and/or operon(s) have been identified, cloned, transformed, transfected or infected into a heterologous organism (or new organism from a synthetic genome), methods known in the art can be used to grow said heterologous organism to produce and purify the desired VOC (e.g. hydrocarbon), including those described herein.

Unless otherwise explained, all technical and scientific terms used herein have the same meaning as commonly understood by one of ordinary skill in the art to which this invention belongs. Definitions of common terms in molecular biology may be found in Benjamin Lewin, Genes IX, published by Oxford University Press, 2007 (ISBN-10 0131439812); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: A Comprehensive Desk Reference, published by VCH Publishers, Inc., 1995 (ISBN 1-56081-569-8); Oxford Dictionary of Biochemistry and Molecular Biology, Revised Edition, 2000.

The singular terms “a,” “an,” and “the” include plural referents unless context clearly indicates otherwise. “Comprising” means “including.” “Comprising A or B” means “including A or B” or “including A and B.” The abbreviation, “e.g.” is derived from the Latin exempli gratia, and is used herein to indicate a non-limiting example. Thus, the abbreviation “e.g.” is synonymous with the term “for example.” It is further to be understood that all base sizes or amino acid sizes, and all molecular weight or molecular mass values, given for nucleic acids or polypeptides are approximate, and are provided for description.

Suitable methods and materials for the practice or testing of the disclosure are described herein. However, the provided materials, methods, and examples are illustrative only and are not intended to be limiting. Accordingly, except as otherwise noted, the methods and techniques of the present disclosure can be performed according to methods and materials similar or equivalent to those described and/or according to conventional methods well known in the art and as described in various general and more specific references that are cited and discussed throughout the present specification (see, e.g., Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; and Harlow and Lane, Antibodies: A Laboratory Manual, Cold Spring Harbor Laboratory Press, 1990 and 1999).

### EXAMPLES

Data disclosed in Examples 1 through 5 were previously published in a report entitled “An Endophytic Gliocladium sp. of Eucryphia cordifolia Producing Selective Volatile Antimicrobial Compounds” (Plant Science 165: 913-922, 2003), which publication is incorporated herein by reference.

### Example 1

### Materials and Methods

This example provides the general materials and methods utilized in Examples 2-5.

### Fungal Isolation and Storage.

Several small limbs of a mature Eucryphia cordifolia located obtained at 41° 32′ 52″ South and 72° 35′ 39″ West were removed and immediately transported by air for processing. In order to select fungi that may produce volatile antibiotics or other biologically active substances, a unique four quadrant Petri plate system was used. PDA was placed in all four quadrants and then Muscodor albus, an endophytic fungus known to produce volatile antibiotics, was placed in one quadrant and allowed to grow for 14 days (Worapong et al. Mycotaxon. 81: 463-475, 2001). Thus, the volatiles of M. albus were being used as a selection tool for other volatile antibiotic producing fungi. Small pieces of the inner bark and outer xylem tissues of E. cordifolia were pretreated with 70% ethanol, flamed and then cambium, phloem and outer xylem tissues were aseptically removed placed in the remaining three quadrants of the plate. After incubation for several days, hyphal tips of developing fungi from the stem pieces were removed and placed on PDA and incubated at 23° C. One particular fungus, designated “isolate C-13”, produced a sour odor and was therefore chosen for future research. In addition, hyphal tips from isolate C-13 were placed on water agar with δ-irradiated carnation leaves (0.5×0.5 cm) in order to encourage spore production. The fungus was deposited as isolate 2259 in the Montana State University mycological culture collection and stored in 15% glycerol at −70° C. The fungus remained viable for at least 4 years, and possibly longer, under these conditions. These deposits were under the control of the inventors, kept confidential and permission to access the deposit was required.

### Scanning Electron Microscopy.

Scanning electron microscopy was performed on isolate C-13 by placing agar pieces and as well as host plant pieces supporting fungal growth into #1 Whatman filter paper packets. The packets were made by folding the filter paper over a piece of cork (1.5 cm). The packets were tied with cotton string and two removable split shot sinkers (ca. 3.25 g each) were attached next to the packets to hold them under the surface of the dehydrating solutions and the liquid carbon dioxide during critical point drying. The fungal preparation was then placed into 2% glutaraldehyde in 0.1 M sodium cacodylate buffer (pH 7.2-7.4) with Triton X, a wetting agent, aspirated for 5 minutes and left overnight. The next day they were washed in 6 changes of water-buffer, followed by three to 15 min. changes in 10% ethanol, four to 15 min. changes of 30% ethanol, five to 15 min. changes of 50% ethanol and left for two days in 70% ethanol. They were then rinsed in five to 15 min. changes of 95% ethanol and then five to 15 min. changes in 100% ethanol. The dehydration process was slowly done to discourage the processes of hyphal shriveling which may occur during rapid dehydration. Ultimately, the fungal material was critically point dried, gold sputter coated and examined with a JEOL 6100 scanning electron microscope (SEM).

### Fungal Growth.

Isolate C-13 was grown on the following media in order to observe cultural morphology and to ascertain which medium supports the maximum production of secondary metabolites: PDA (potato dextrose agar), CMA (corn meal agar), OMA (Oatmeal Agar), LBA (Lima Bean Agar), and NPDA (natural PDA). Natural PDA (1 L) was made with 10 g of potato starch, 15 g sucrose, 15 g agar, and distilled water. Growth was measured at 5, 10, 15, and 25 days; fungal morphology was described after the colony had grown for 25 days.

### Volatile Antibiotic Assays.

In order test for the production of antibiotics, isolate C-13 was plated on one half of a Petri plate containing PDA and grown for three weeks at 25° C. (half-plate method) (Strobel et al., Microbiology 147: 2943-2950, 2001). A 5 mm plug (obtained with a No. 1 cork borer) of the test organism was then placed on the plate and growth was recorded after 3 and 7 days of exposure. Then, if no growth was observed on the 5 mm plug (containing the test microbe), it was transferred to a Petri plate of PDA and checked for viability. The following test organisms were used in these studies: Rhizoctonia solanii, Verticillium dahliae, Pythium ultimum, Geotrichum candidum, Fusarium oxysporum, Aspergillus ochraceus, Gliocladium viriens, and Sclerotinia sclerotiorum. Furthermore, a bioassay system (plate to plate method) was devised for testing volatile antibiotic production wherein the isolate C-13 was plated on the desired medium and grown for a given number of days. Then, a fresh plate of PDA was physically attached to the plate containing the test organism and the two plates sealed with two layers of parafilm. Measurements of the growth of the test fungus (initially acquired from agar plate with a No. 1 cork borer, yielding a 5 mm plug) were made at appropriate intervals. However, a modification of this bioassay was devised in which the test plate was first fumigated by the volatiles of isolate C-13 for 7 days, at which time a 5 mm plug of the test organism was placed on the fumigated PDA plate. Again, the two plates (C-13 culture and the plate containing the test organism) were attached and sealed together and growth was measured in colony diameter in mm at 3 and 7 days. This bioassay modification allowed for a greater time exposure to the volatile antibiotics and gave more significant results. Data are presented as percentage of growth compared to a control or untreated test fungus.

### Quantitative and Qualitative Analyses of Volatile Organic Substances.

The gases in the air space above the C-13 mycelium growing in Petri plates were most specifically analyzed by trapping the fungal VOCs with a “Solid Phase Micro Extraction” syringe. The fiber material (Supelco) was 50/30 divinylbenzene/carburen on polydimethylsiloxane on a stable flex fiber. The syringe was place through a small hole drilled in the side of the Petri plate and exposed to the vapor phase for 45 minutes. The syringe was then inserted into a gas chromatograph (Hewlett Packard 5890 Series II Plus) equipped with a mass-selective detector. A 30 m×0.25 mm I.D. ZB Wax capillary column with a film thickness of 0.50 mm was used for the separation of the volatiles. The column was temperature programmed as follows: 25° C. for 2 minutes followed by 220° C. at 5° C./minute. The carrier gas was Helium Ultra High Purity (local distributor) and the initial column head pressure was 50 kPa. The helium pressure was ramped with the temperature ramp of the oven to maintain a constant carrier gas flow velocity during the course of the separation. Prior to trapping the volatiles, the fiber was conditioned at 240° C. for 20 minutes under a flow of helium gas. A 30-second injection time was used to introduce the sample fiber into the gas chromatograph that was interfaced to a VG 70E-HF double focusing magnetic mass spectrometer operating at a mass resolution of 1500. The MS was scanned at a rate of 0.50 seconds per mass decade over a mass range of 35-360 amu. Data acquisition and data processing was performed on VG SIOS/OPUS interface and software package. Initial identification of the unknowns produced by isolate C-13 was made through library comparison using the NIST database. Control analyses were conducted using Petri plates containing only PDA. The compounds obtained therefrom, mostly styrene, were subtracted from the analyses done on Petri plates containing PDA and isolate C-13. Final identification and confirmation of many of the compounds was done by acquiring them from commercial sources or making them via organic syntheses and subjecting them to GC/MS done in an exact manner as indicated above (Strobel et al., Microbiology 147: 2943-2950, 2001). Those in this category are appropriately indicated in the GC/MS analysis.

The second method involves the proton transfer reaction-mass spectrometry (PTR-MS). Basically, the PTR-MS instrument ionizes organic molecules in the gas phase through their reaction with H₃O⁺, forming mostly protonated molecules (MH⁺ where M is the neutral organic molecule) which can then be detected by a standard quadrupole mass spectrometer. This process can be run on real air samples with or without dilution, since the primary constituents of air (nitrogen, oxygen, argon, and carbon dioxide) have a proton affinity less than water and thus are not ionized. Most organic molecules (excepting alkanes) have a proton affinity greater than water and are therefore ionized and detected. A further advantage of PTR-MS is that from the known or calculated quantities; the reaction time, the amount of H₃O⁺ present, and the theoretical reaction rate constant for the proton transfer reaction, the absolute concentration of constituents in a sample can be quantified (Lindinger, W., et al. (1998) Internat J Mass Spectrometry Ion Proc., 173, 191-241). Finally, an enormous advantage of PTR-MS is that it can be run in real time and continuously produce data on the concentrations of specific ions of interest.

### Assay of the Artificial Mixtures of the Volatiles of Fungal Volatiles.

An artificial mixture of compounds, as well as [8]annulene, that were positively identified in the atmosphere of the Gliocladium sp., were subjected to PDA plate bioassay tests for a two day exposure period. They were available from Sigma-Aldrich (St. Louis, Mo.) or were prepared via organic synthetic techniques (Strobel et al., Microbiology 147: 2943-2950, 2001). Varying amounts (non-equilibrium conditions) of the mixtures of the VOCs (0.8-30 μl) were placed in a small sterile plastic microcup (4×6 mm) that was firmly fixed in the middle of the test plate, and then test organisms were inoculated around the periphery of the agar surface. The amount of each VOC used was calculated from relative areas (RA's) in Table 2. For instance, 1-octene has an RA of 0.23, while 1,3,5,7-cyclooctatetraene has a value of 100, thus the amounts (volumes) of each of these compounds used in the text mixture were 0.23:100 on a volume: volume basis (Table 2). Measurements of fungal growth were made and compared to growth on a control PDA plate having no test compounds. The IC₅₀ and IC₁₀₀ values were calculated from data plots of inhibition of fungal growth as a function of VOC concentrations (Strobel et al., Microbiology 147: 2943-2950, 2001). The IC values are presented as the amount of VOCs (volume per ml of air space in the Petri plate above the fungal culture). Studies were done in triplicate and the data averaged and standard deviations determined (Strobel et al., Microbiology 147: 2943-2950, 2001). Each test organism, after the two day exposure was tested for its viability by placing it on a fresh plate of PDA.

### Fungal DNA Isolation.

Isolate C-13 was grown on PDA in a 9 cm Petri plate for 21 days at 25° C. The mycelium was scraped directly from the surface of the agar culture and weighed. White quartz sand (3 g per g of tissue), phenol/chloroform/isoamyl alcohol 25:24:1 (0.5 ml per g of tissue), and extraction buffer (100 mM Tris HCl pH=8.0, 20 mM EDTA, 0.5M NaCl and 1% SDS at the rate of 0.4 ml per g of sand) were added to the tissue in a mortar and ground vigorously for 30 sec. with a pestle. Extraction buffer was again added (2 ml per 0.5 g of starting tissue) as well as the phenol/chloroform/isoamyl alcohol mixture (1 ml per 0.5 g of starting tissue). After mixing well, the solution was transferred into microfuge tubes and centrifuged at 16,000×g for 5 minutes at 25° C. The aqueous phase was transferred to a new tube and mixed with 0.6 vol. of isopropanol. The sample was incubated at 25° C. for 10 min. and then centrifuged at 4° C. for 15 minutes at 16,000×g to recover the precipitate. The pellet was rinsed with 95% ethanol and then air dried. The pellet was then resuspended in 340 μl TE containing RNase A (20 μg/ml). The sample was incubated at 37° C. for 30 minutes. After incubation 0.3 ml of phenol/chloroform/isoamyl alcohol mixture was added to the sample. The sample was mixed and centrifuged at 4° C. for 2 minutes. The supernatant liquid was transferred to a new tube; 50% vol 7.5 M ammonium acetate and 2.5 volumes of 100% ethanol were added. The samples were incubated for 30 min. at −20° C. and then centrifuged at 4° C. for 15 min at 16,000×g. The pelleted DNA was rinsed with 70% ethanol, air dried, and resuspended in 100 μl ddH₂0. Agarose gel electrophoresis and a UV spectrophotographic system were used to record the data according to the methods of Weiland (Weiland J J, Fungal Genetics Newsletter 44: 60-63).

Amplification of 18 S rDNA.

Partial nucleotide base pair fragments of the 18S rDNA gene from isolate C-13 were amplified via the polymerase chain reaction (PCR) as a single fragment with the primer NS1 (5′ GTA-GTC-ATA-TGC-TTG-TCT-C′3; SEQ ID NO: 1) and NS8 (5′ TCC-GCA-GGT-TCA-CCT-ACG-GA 3′; SEQ ID NO: 2). PCR was performed in a 25 μl reaction vial containing 0.1 μg genomic DNA, 10 mM of each primer, 3 mM of the 4 dNTPs and 0.5 unit Taq polymerase (Fisher) in a 10× Taq buffer A (Fisher) containing 500 mM potassium chloride, 15 mM magnesium chloride, 100 mM Tris-HCL (pH 9.0 at 25° C.) (Table 2). The following cycle parameters were maintained: 95° C. for 5 minutes followed by 34 cycles of 40 seconds at 95° C., 40 seconds at 45° C. and 40 seconds at 72° C. followed by 5 minutes at 72° C.

Amplification of Internal Transcribed Space Sequences (ITS) and 5.8S rDNA.

The ITS regions of the test fungus were amplified using PCR and the universal ITS primers ITS1 (5′ TCC-GTA-GGT-GAA-CCT-GCG-G 3′; SEQ ID NO: 3) and ITS4 (5′ TCC-TCC-GCT-TAT-TGA-TAT-GC 3′; SEQ ID NO: 4) (Table 2 below) (White et al., In PCR Protocols: A Guide to Methods and Applications, Ed. M. A. Innis, Gelfand, Sninsky and White, Academic Press, Inc., NY, pp. 315-322, 1990). PCR was performed using the same cycle parameters described previously with the exception that annealing temperature was 60° C. The PCR products were purified and desalted using the QIAquick PCR purification kit (Qiagen, Valencia, Calif.).

### Cloning.

The PCR product was cloned into a pDrive TA vector (Qiagen PCR cloning kit) according to manufacturer's instructions.

### Transformation and Extraction.

Preparation of competent cells was performed by the CCNB80 method. DH5a E. coli were grown to 0.3 OD at 595 nm. The culture was chilled on ice for 10 min. The cells were then pelleted by centrifugation for 10 min. 4° C. at 5000 rpm. The pellet was resuspended in ⅓ of the original volume of cold CCNB80 (for 1 liter-10 ml KAct, 2 g MgCl₂, 4 g MnCl₂, 11.8 g CaCl₂, 100 ml glycerol). This suspension was left on ice for 20 minutes, repelleted, and resuspended in CCNB80 ( 1/12 the original volume). This suspension was incubated on ice for 10 min and then divided into 200 μl aliquot Eppendorf vials and immediately frozen in −80° C. The DNA transformation into the cells was performed according to standard procedures (Ausubel et al., Current Protocols in Molecular Biology, Mass. General Hospital of Harvard Medical School, Cambridge, Mass., 1998). The transformed cells were plated on LB agar supplemented with 30 μg/ml kanamycin sulfate (Sigma-Aldrich, St. Louis, Mo.), in the presence of IPTG and X-gal for blue/white selection. White single colonies were grown in LB broth and DNA was extracted using a Perfectprep Plasmid Mini (Eppendorf) according to manufacturer's instructions. Presence of the insert was confirmed by DNA digestion with EcoRI restriction enzyme (Promega).

Cycle Sequencing 18S Ribosomal DNA, ITS Regions and 5.8S rDNA.

The plasmid inserts were sequenced by the Plant-Microbe Genomics Facility at Ohio State University using an Applied Biosystems 3700 DNA Analyzer and BigDye™ cycle sequencing terminator chemistry and the universal primers T7 and Sp6 and 4Sp61/4T71 for 18S as internal primers (Table 2, below).

TABLE 2

Primers used to determine 18S rDNA, ITS and
5.8S nucleotide sequences of Gliocladium sp.

| | | |
|---|---|---|
| Primers | SEQ ID NO | Sequences from 5′ to 3′ |

| | | |
|---|---|---|
| NS1 | SEQ ID NO:1 | 5′ GTAGTCATATGCTTGTCTC′3 |
| NS8 | SEQ ID NO:2 | 5′ TCCGCAGGTTCACCTACGGA 3′ |
| ITS1 | SEQ ID NO:3 | 5′ TCCGTAGGTGAACCTGCGG 3′ |
| ITS4 | SEQ ID NO:4 | 5′ TCCTCCGCTTATTGATATGC 3′ |
| SP6 | SEQ ID NO:5 | 5′ CATTTAGGTGAACACTATAG 3′ |
| T7 | SEQ ID NO:6 | 5′ GTAATACGACTCACTATAG3′ |
| 4SP61 | SEQ ID NO:7 | 5′ GCCTTTCCTTCTGGGGAGCATG 3′ |
| 4T71 | SEQ ID NO:8 | 5′ CTGATCGTCTTCGATCCCCTAAC 3′ |

### Example 2

### Identification of the Endophytic Fungal Isolate C-13

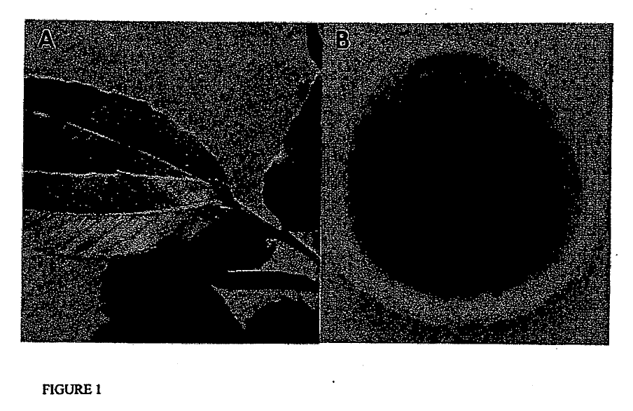

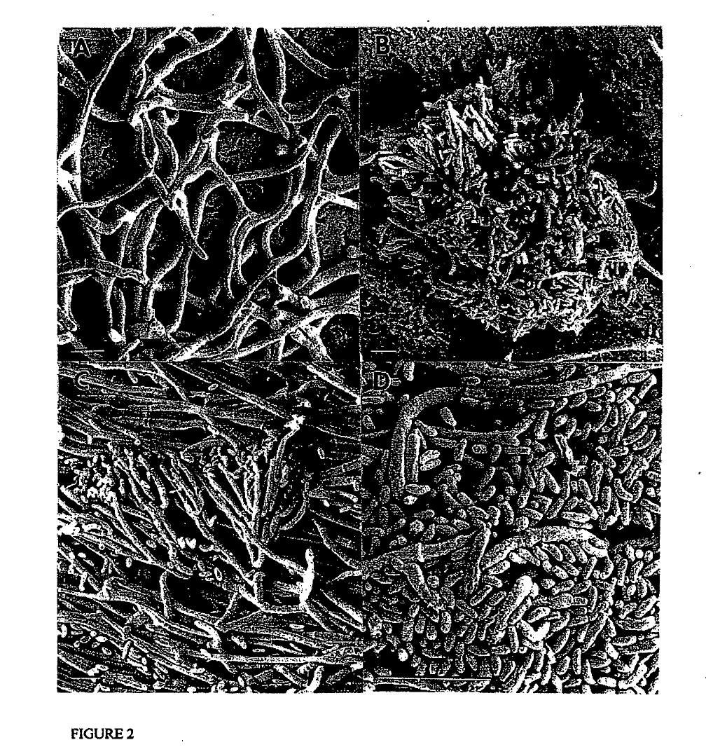

This example describes the identification of the endophytic fungal isolate C-13. A number of endophytic fungi were isolated from E. cordifolia, but the one of greatest interest was labeled “isolate C-13” (FIGS. 1A and 1B). This organism grew well on each medium that was tested. It produced a mycelium with concentric rings on CMA, while doing the same on NPDA and LB, but having pinkish and yellowish colorations, respectively on these media. On OMA, fungal growth was powdery, and variously colored. On PDA, however the mycelia developed a whitish powdery character. The mycelium gradually, within a week, became fluffy and began to display colors varying from powdery pink to purple to olive-toned. Blackish to purplish somewhat spherical-like bodies began to be deposited at the edge of the mycelium (FIG. 1B). The fungus produces eliposoidal, polysymmetical conidiospores ranging in size from ca. 1.8×5.0 μm on phialides ranging from 2 dia×10-15 μm in length (FIGS. 2A-2D). The conidial masses of the fungus are round, whitish, and slimy. Isolate C-13 answered the majority of the descriptors of the fungal genus—Gliocladium—which is referred to as Gliocladium sp. Organisms of this type are known as both saprophytes, as well as pathogens of plants, but not generally known as endophytes (Schroers et al., Mycologia 91: 365-385, 1999; Redlin and Carris, Endophytic Fungi in Grasses and Woody Plants, APS Press, St. Paul, Minn., 1996).

This organism was further characterized via molecular techniques. Its 5.8S, ITS 1 and ITS2 regions were isolated, cloned and BLASTED to the NCBI-GenBank showing 92% homology to Chloroscypha enterochroma, an organism structurally unrelated to Gliocladium spp. These sequences are entered in GenBank as AY221904. The 18S rDNA sequence shows the highest relatedness to an uncultured eukaryote and it is entered as GenBank AY219040. The molecular structural characteristics of Gliocladium sp. (isolate C-13) do not correspond well with the GenBank database. One explanation may be that there are not yet significant molecular data in GenBank for meaningful comparisons to be made for this fungal genus.

### Example 3

### Biological Activity of Gliocladium sp. in Volatile Antibiotic Assays

This example illustrates the biological activity of Gliocladium sp. in volatile antibiotic assays.

In the initial standard bioassay tests, using the half plate method, among the test organisms representing a series of plant pathogens, P. ultimum and V dahaliae were strongly inhibited by the gases of Gliocladium sp. for this reason, the half plate bioassay system on 16 and 19 day old cultures of Gliocladium sp. were used. Further, organisms that gave the best responses to the volatile compounds in the air space above the agar were evaluated. A 40.9±6.6 percent reduction in growth of P. ultimum on PDA (relative to the control) was observed on a 16 day old culture of Gliocladium sp. after a 1 day exposure to the fungal VOCs. Then, after 5 and 7 days of incubation, there was 36±10.4% and 35.8±10.4% inhibition in the growth of P. ultimum, respectively. Comparable results were obtained when a 19 day old Gliocladium sp. culture was tested, with a substantial inhibition (58±7.3%) occurring only after 1 day of incubation of P. ultimum on the test plate. Studies with Gliocladium sp. grown on OMA gave similar results relative to inhibition of test organisms. While the test fungus is inhibited, the critical events in inhibition occur after a day or so of exposure to the volatile compounds. The fungus remains viable, but continues to grow slowly relative to the growth of the control (untreated test fungus). It appears that inhibitory factor(s) are present and the test fungus does not overcome their influence. Also, it may be the case that the inhibitors are not present in great enough concentrations to be lethal to the test fungus.

Thus, in order to ascertain if greater biological influences of the fungal VOCs can be measured more accurately on other test fungi, a modified plate to plate method was devised. In this system, the test plate is first exposed to the gases of Gliocladium sp. for 7 days and then the test organism placed on the test plate and the plates resealed with parafilm. The results showed that the fungi having the greatest sensitivity to the Gliocladium sp. VOCs, after 3 and 7 days of incubation on the test plate were Pythium ultimum and Verticillium dahliae, however Sclerotinia sclerotiorum, Rhizoctonia solani, Geotrichm candidum, and Aspergillus sp. were also sensitive to these compounds (Table 3), while Fusarium oxysporum was virtually resistant (Table 3). Interestingly, enough, both Pythium ultimum and Verticillum dahliae were killed in this assay test when the plates had a pre-exposure to Gliocladium sp. (Table 3). Certain aspects of these observations are comparable to those made on Muscodor albus in that Fusarium spp. are generally resistant to volatile antibiotics of this and related organisms (Strobel et al., Microbiology 147: 2943-2950, 2001). Generally, however, a prolonged incubation of the test plate (7 days) there was an increase the relative inhibitory effect with virtually all of the test organisms except Gliocladium virens. This organism, although initially showing inhibition, seemed to overcome the effect with time (Table 3). A repeat of the entire experiment yielded comparable results after the Gliocladium sp. had been grown for 19 days prior to the 7 day PDA plate exposure period.

Gliocladium sp. was cultured for 11 days on PDA at which time a fresh PDA plate was placed on top (face to face) of the PDA plate and sealed with two layers of parafilm. After a 7 day fumigation period at 23° C. the test organism was placed on the fumigated PDA plate, and the plate was replaced to its original position sealed to the PDA plate containing Gliocladium sp. and growth measurements made after 3 and 7 days. Then, after one week, the test organism was transferred to a fresh PDA plate in order to observe its viability.

TABLE 3
The influence of the VOCs of Gliocladium sp. on other fungi.

| | | Inhibition % | |
|---|---|---|---|
| | Inhibition % over | over control at | Viability |
| | control at 3 days | 7 days | after 1 |
| Test Organism | incubation | incubation | week |
| Sclerotinia sclerotiorum | 10.8 ± 8 | 31.7 ± 28 | Alive |
| Rhizoctonia solani | 12.2 ± 8 | 30.7 ± 7 | Alive |
| Geotrichum candidum | 16.6 ± 2 | 44.4 ± 3 | Alive |
| Pythium ultimum | 100 | 100 | Dead |
| Verticillium dahliae | 100 | 100 | Dead |
| Gliocladium virens | 50 ± 6 | 9 ± 9 | Alive |
| Fusarium oxysporum | 3.3 ± 2 | 17.5 ± 5 | Alive |
| Aspergillus ochraceus | 43.0 ± 4 | 65.3 ± 2 | Alive |

### Example 4

### Chemical Composition of the Disclosed Volatiles

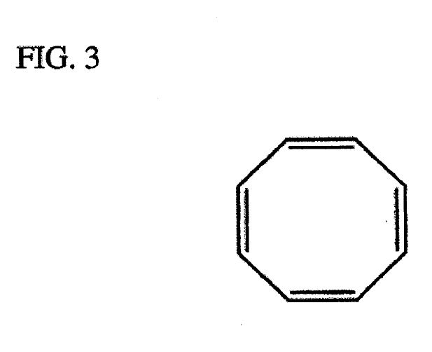

This example illustrates chemical composition of the disclosed volatiles. The inhibitory effect of Gliocladium sp. against a variety of different fungi is unequivocally due to volatile compounds that it produces in culture. Gas trapping and analytical experiments were done to ascertain the chemistry of the volatiles produced by this fungus. The volatile compounds were tentatively identified on the basis of the NIST data base by virtue of comparisons made of the actual mass spectral data acquired on each compound to the data base. Final identification of some of the volatiles was done by using authentic standard compounds that had been acquired from commercial sources or chemically synthesized and analyzed under the same GC/MS conditions (Strobel et al., Microbiology 147: 2943-2950, 2001). Some of the same microbial inhibitory compounds produced by Muscodor albus are also produced by Gliocladium sp., including phenylethyl alcohol, acetic acid, 2-phenylethyl ester, and 1-propanol, 2-methyl-(Strobel et al., Microbiology 147: 2943-2950, 2001). However, the volatile compound produced in the greatest amount by Gliocladium sp. was positively identified as 1,3,5,7-cyclooctatetraene ([8]annulene) (FIG. 3). There appears to be no reports of [8]annulene either as an endophytic microbe product or as a fungal product in general (McAfee and Taylor, Natural Toxins 7: 283-303, 1999). An earlier review indicates that that none of the annulenes have ever been discovered as products of living organisms (March, Advanced Organic Chemistry 3^{rd} Ed., Wiley and Sons, NY, 1984). Until now, this fact continues to be born out by a comprehensive literature search. One of the most interesting aspects of [8]annulene is that it is an anti-aromatic compound, unstable, flammable, explosive, and having such properties allowed for its use as a rocket propellant during the second World War.

Other octane derivatives appeared in the analysis of the atmosphere of Gliocladium sp. including 1-octene, 1,3-octadiene, 3-octanone and 7-octen-4-ol. Several other octane derivatives have been tentatively identified in Cantharellus cibarius, however both 1-octene and 3-octanone were positively identified in Gliocladium sp. (McAfee and Taylor, Natural Toxins 7: 283-303, 1999). Other volatile compounds, likewise, were not available so they could not be firmly identified and tested for their individual or combined biological effects.

### Example 5

### Evaluating Artificial Mixtures of the Fungal Volatiles

This example illustrates the evaluation of artificial mixtures of fungal volatiles. In order to provide unequivocal evidence for the involvement of volatile organic substances as the source of the fungal inhibition and the killing phenomenon, an artificial mixture of the positively identified volatile compounds was prepared and evaluated. Each compound was added to the mixture in a ratio with respect to the relative area (RA) of the fungal volatiles (Table 4).

The atmosphere of a 19 day old Gliocladium sp. culture growing on PDA was analyzed using the gas trapping and GC/MS methods as described in Example 1.

TABLE 4
A GC/MS analysis of the VOC's of Gliocladium sp.

| | Time | | |
|---|---|---|---|
| Compound Identified | (Min.) | MW | RA^{#} |
| *1-Butanol, 3 methyl | 1:34 | 88 | 0.29 |
| *1-Octene | 3:41 | 112 | 0.23 |
| 1,3-Octadiene | 6:21 | 110 | 0.34 |
| Furan, 2,5 dimethyl | 6:30 | 96 | 0.34 |
| Butanoic acid, 3-methyl-, methyl ester | 8:17 | 116 | 3.45 |
| Propanoic acid, 1-methylpropyl ester | 9:11 | 130 | 0.47 |
| 3-Hexanone, 4-methyl- | 9:51 | 114 | 1.16 |
| *1-Propanol, 2-methyl- | 10:39 | 74 | 1.06 |
| 4-Decene, 9-methyl- | 12:07 | 154 | 0.25 |
| 2-Pentanol, 4-methyl- | 12:40 | 102 | 1.47 |
| *1-Butanol, 2-methyl- | 13:52 | 88 | 20:34 |
| Hexanoic Acid, 2,4-dimethyl-, methyl ester | 14:25 | 158 | 0.51 |
| *Propanoic Acid | 14:43 | 144 | 0.25 |
| *3-Octanone | 15:11 | 128 | 0.76 |
| 3-Hexanol, 4-methyl- | 15:16 | 116 | 6.10 |
| *1,3,5,7-Cyclooctatetraene | 15:22 | 104 | 100.00 |
| 2-propanol, 1-1′-[(1-methyl-1,2 ethanediyl) | 15:32 | 192 | 0.48 |
| bis(oxy)]bis- | | | |
| 2-Butanol, 3,3-dimethyl- | 16:21 | 102 | 1.94 |
| 7-Octen-4-ol | 20:34 | 128 | 1.17 |
| 1H-Cycloprop[e] azulene 1a,2,3,4,4a,5,6,7b- | 22:12 | 204 | 0.37 |
| octahydro-1,1,4,7-tetramethyl- | | | |
| *Acetic acid, 2-phenylethyl ester | 29:29 | 164 | 4.41 |
| *Phenylethyl Alcohol | 31:31 | 122 | 6.83 |

*Positively identified by virtue of the passage of a standard known compound through the GC/MS system under identical conditions with identical results to the analysis of the volatiles of the fungal gases as outlined in Example 1.
^{#}RA = relative peak area (%). Compounds not giving at least a relative value of 0.2 were not tallied on this table.

The test fungi were exposed to amounts of the VOC's varying from 0.8 μl to 30 μl (non-equilibrium conditions in a 50 ml air space volume above the fungal culture on the Petri plate) and exposed to them for two days after which growth rates were measured and the IC₅₀'s and IC₁₀₀'s calculated. In general, each test fungus was inhibited by the artificial VOC mixture which was also true for the effects of the Gliocladium sp. atmosphere on the same fungi (Tables 2 and 4). There appeared to be no relationship, however between lower IC₅₀ and IC₁₀₀ values and the ultimate viability of the test fungus (Table 5). Furthermore, the IC₅₀ values for the test fungi were in the same concentration range as previously observed for the artificial atmosphere of M. albus (Strobel et al., Microbiology 147: 2943-2950, 2001). The majority of the fungi responded in the same manner as they did in the presence of Gliocladium sp. atmosphere, that is P. ultimum died in both cases, while the others had been only inhibited (Tables 3 and 5). Nevertheless, while V. dahliae died in the presence of Gliocladium sp., it survived in the artificial atmosphere. Just the reverse was true for S. sclerotiorum (Tables 3 and 5). Furthermore, Gliocladium sp. itself was inhibited by the artificial atmosphere at a relatively low IC₅₀ value, but it was not killed by the VOCs (Table 3). Similar results were observed when M. albus was tested in its own artificial atmosphere (Strobel et al., Microbiology 147: 2943-2950, 2001).

Also, while [8]annulene is the most abundant of the volatiles of Gliocladium sp., it is extremely bioactive as an inhibitor of the growth of test fungi. The IC₅₀'s of this compound varied from 0.05 to 0.28 μl/ml and nearly matched the values of the artificial mixture itself (Tables 5 and 6). This suggests, that [8]annulene is both the most abundant VOC in the atmosphere of Gliocladium sp., and is also the compound with the greatest biological activity. Likewise, the IC₁₀₀ values for [8]annulene approximated those of the complete artificial mixture, but under no identical test conditions were these values identical (Tables 5 and 6). Furthermore, P. ultimum and S. sclerotiorum did not die after exposure to [8]annulene alone which is not the result obtained with the artificial mixture, indicating that one or more of the other ingredients in the artificial mixture contributes to cell death, probably in a synergistic manner (Tables 5 and 6).

The results show the growth of test fungi after the exposure to an artificial mixture of the identified VOC's found in the atmosphere of Gliocladim sp. for two days. The artificial mixture was prepared based on the ratios of the compounds identified by GC/MS. The IC₅₀'s were calculated on the basis of the μl VOCs/ml of the free gas space in the Petri plate above the fungal culture. The IC₁₀₀'s were extrapolated from the curves obtained by plotting fungal inhibition vs. VOC concentration. The IC₁₀₀'s represent the approximate concentration required to give 100% inhibition of fungal growth. Viability was evaluated by transferring the fungal to a regular PDA plate after exposure to the VOC mixtures.

TABLE 5
The influence of an artificial mixture of Gliocladium sp.
VOCs on various fungi.

| Test Organism | IC₅₀ μl/ml | IC₁₀₀ μl/ml | Viability |
|---|---|---|---|
| Sclerotinia sclerotiorum | 0.05 ± 0.01 | 0.18 | dead |
| Rhizoctonia solani | 0.14 ± 0.08 | 0.60 | alive |
| Geotrichum candidum | 0.10 ± 0.02 | 0.60 | alive |
| Pythium ultimum | 0.10 ± 0.04 | 0.44 | dead |
| Verticillium dahliae | 0.12 ± 0.03 | 0.60 | alive |
| Fusarium oxysporum | 0.28 ± 0.03 | 0.66 | alive |
| Aspergillus ochraceus | 0.08 ± 0.01 | 0.30 | alive |

* Viability was determined by placing the agar blocks containing the test fungi, after exposure to the VOC's at the IC₁₀₀ level, on a fresh agar plate and observing growth or no growth of the test organism.

The IC₅₀'s were calculated on the basis of the μl [8]annulene/ml of the free gas space in the Petri plate above the fungal culture. The IC₅₀'s and IC₁₀₀'s were calculated in the same manner as described in Example 1. The IC₁₀₀'s represent the approximate concentration required to give 100% inhibition of fungal growth. Viability was tested by transferring the fungal to a regular PDA plate after exposure to [8]annulene.

TABLE 6
A test on inhibition and lethality of various test fungi
after a two day exposure to [8]annulene.

| Test Organism | IC₅₀ μl/ml | IC₁₀₀ μl/ml | Viability |
|---|---|---|---|
| Sclerotinia sclerotiorum | 0.05 ± 0.01 | 0.30 | alive |
| Rhizoctonia solani | 0.14 ± 0.08 | 0.40 | alive |
| Geotrichum candidum | 0.10 ± 0.02 | 0.44 | alive |
| Pythium ultimum | 0.10 ± 0.04 | 0.30 | alive |
| Verticillium dahliae | 0.18 ± 0.00 | 0.30 | alive |
| Fusarium oxysporum | 0.28 ± 0.03 | 0.70 | alive |
| Aspergillus ochraceus | 0.08 ± 0.01 | 0.50 | alive |
| Gliocladium sp. | 0.30 ± 0.01 | 0.60 | alive |

* Viability was determined by placing the agar blocks containing the test fungi, after exposure to [8]annulene at the IC₁₀₀ level, on a fresh agar plate and observing growth or no growth of the test organism.

In summary, the discovery of endophytic fungi that make potent and biologically specific volatile antibiotics and other bioactive volatiles is a relatively new occurrence (Worapong et al., Mycotaxon. 81: 463-475, 2001; Worapong et al., Mycotaxon. 79: 67-79, 2001; Strobel et al., Microbiology 147: 2943-2950, 2001). Thus far, all of these organisms have clustered into a very tight taxonomic group—Muscodor spp. according to molecular analyses (Worapong et al., Mycotaxon. 81: 463-475, 2001; Worapong et al., Mycotaxon. 79: 67-79, 2001; Strobel et al., Microbiology 147: 2943-2950, 2001; Daisy et al., Microbiology 148: 3737-3741, 2002). Although wood associated fungi are known to make biologically active volatile substances, none have been demonstrated to make suites of molecules having the impressive power to specifically inhibit and kill competing microbes, as does M. albus and M. roseus (Worapong et al., Mycotaxon. 81: 463-475, 2001; Worapong et al., Mycotaxon. 79: 67-79, 2001; Strobel et al., Microbiology 147: 2943-2950, 2001; Daisy et al., Microbiology 148: 3737-3741, 2002). As a result of this work, an entirely different fungal genus—Gliocladium sp. has been shown to produce a different suite of volatile compounds having both inhibitory and lethal properties to other microbes. The killing effect of the VOCs of this organism are only observed in P. ultimum and V. dahliae which is an unusual biological response given the fact that these two organisms do not share a common taxonomic basis (Table 3). It would seem that some endophytes may use the mechanism of volatile antibiotic production to contribute to the well being of the host plant by offering it protection from invading pathogens by virtue of volatile antibiotics. However, only circumstantial evidence exists for this concept and it might be an important natural phenomenon. Furthermore, there is an interesting prospect that mixtures of the VOC's that mimic the concentration and identity of the ingredients of the gas producing fungi may prove useful by themselves in agricultural, industrial, and military applications. For instance, most recently the Muscodor spp. has been tested for use in certain agricultural settings to treat pathogen-infested soils, plants, and seeds (Strobel et al., Microbiology 147: 2943-2950, 2001).

Some applications of these fungi for practical uses may be limiting since certain pathogenic organisms are not as sensitive to the Muscodor spp. VOCs as others. Thus, a search for other endophytic volatile antibiotic producers seems warranted. This study demonstrates that another effective gas producer is a Gliocladium sp. and although its biological effects against various fungal pathogens is not as great as M. albus or M. roseus, at least it can be understood that the phenomenon of specific volatile antibiotic production by endophytes is not limited to only one genus—Muscodor spp.

### Example 6

### Production of Hydrocarbons by Gliocladium sp

This example illustrates the production of hydrocarbons by Gliocladium sp. Gliocladium sp was also grown on a number of individual media containing various carbon sources such as: glycerol, cellulose, barley, potato dextrose (PD), soybean and oatmeal agars, in order to characterize the ability of this organism to grow on these agars. Excellent growth was noted on oatmeal, barley and potato agar bases with reasonable growth on the cellulose medium. The organism, on the PDA medium, produces some renewable energy sources, such as 1-octene, 1,3-octadiene, 3-octanone and others such as [8]annulene or 1,3,5,7-cyclooctatetraene (FIG. 3).

In order to learn if the quantity and quality of important hydrocarbons might be changed, Gliocladium sp. was grown microaerophilically on the oatmeal agar (Difco) for 10 days. The microaerophilic conditions (reducing without the presence of oxygen) may be favorable to the production of reduced compounds such as alkanes and cyclic alkanes.

The organism was grown in a 250 ml amber bottle made of borosilicate glass with a PTFE stopper that resulted in a perfect seal after the organism was inoculated on to a slant of 80 ml of oatmeal agar. Incubation was a 23° C. for 10 days after which, a baked “Solid Phase Micro Extraction” syringe (Supelco) consisting of 50/30 divinylbenzene/carburen on polydimethylsiloxane on a stable flex fiber was placed through a small hole made in the stopper and it was exposed to the vapor phase for 45 minutes. The syringe was then inserted into the splitless injection port of a Hewlett Packard 6890 gas chromatograph containing a 30 m×0.25 mm I.D. ZB Wax capillary column with a film thickness of 0.50 mm. The column was temperature programmed as follows: 30° C. for 2 minutes followed to 220° C. at 5° C./minute. The carrier gas was ultra high purity Helium and the initial column head pressure was 50 kPa. Prior to trapping the volatiles, the fiber was conditioned at 240° C. for 20 minutes under a flow of helium gas. A 30 second injection time was used to introduce the sample fiber into the GC. The gas chromatograph was interfaced to a Hewlett Packard 5973 mass selective detector (mass spectrometer) operating at unit resolution. The MS was scanned at a rate of 2.5 scans per second over a mass range of 35-360 amu. Data acquisition and data processing were performed on the Hewlett Packard ChemStation software system. Initial identification of the unknowns produced by Gliocladium sp. was made through library comparison using the NIST database.

A similar operation was conducted with a bottle containing only oatmeal agar as a control. All data obtained from the control bottle were subtracted from those in the bottle containing the Gliocladium sp. The data in Table 7 show that there are many more new hydrocarbons, esters, acids and alcohols in the oatmeal agar, microaerophilic conditions than on the PDA plate under Petri plate conditions. Many of these compounds are commonly found in hydrocarbon fuels, including 1-octene, heptane, 2-methyl, hexadecane, undecane, the decane derivatives and the cyclic compounds. Traces of octane, ethanol, and 2-n-Butyl furan were also detected in the control bottle.

TABLE 7
A GC/MS air space analysis of the volatile compounds produced by G. roseum after an
18 day incubation under microaerophilic conditions at 23° C. on medium A (oatmeal agar).

| Time | Relative | | | Molecular |
|---|---|---|---|---|
| (retention) | Area | Stds *+♦ | Possible Compound [symbol] | Weight |
| 1.603 | 5.4 | ♦ | Oxirane, ethyl- | 72.06 |
| 2.081 | 3.4 | *+ | Heptane, 2-methyl- [symbol] | 114.14 |
| 2.666 | 14.1 | *+ | Octane [symbol] | 114.14 |
| 3.138 | 15.5 | *+ | 1-Octene [symbol] | 112.13 |
| 4.598 | 24.5 | * | Ethanol | 46.04 |
| 4.872 | 1.2 | * | Cyclohexene, 4-methyl- | 96.09 |
| 5.204 | 13.5 | ♦ | Hexane, 2,4-dimethyl-(possible isomer) | 114.14 |
| 5.378 | 10.8 | | Undecane, 2,6-dimethyl- | 184.22 |
| 5.533 | 13.5 | ♦ | Hexadecane [symbol] (possible isomer) | 226.27 |
| 5.941 | 10.5 | | Heptane, 5-ethyl-2,2,3-trimethyl-(or isomer) | 170.20 |
| 6.365 | 8.7 | + | Undecane, 4-methyl- [symbol] | 170.20 |
| 6.418 | 5.4 | | Heptane, 5-ethyl-2,2,3-trimethyl-(or isomer) | 170.20 |
| 6.668 | 6.1 | | Octane, 3-ethyl-2,7-dimethyl- | 170.20 |
| 6.768 | 12.1 | | Decane, 2,2,6-trimethyl- | 184.22 |
| 6.931 | 6.8 | *+ | Undecane [symbol] | 156.19 |
| 7.112 | 5.4 | | Decane, 3,3,5-trimethyl- | 184.22 |
| 7.173 | 6.4 | * | Nonane, 3-methyl- [symbol] | 142.17 |
| 7.232 | 7.3 | * | 1-Propanol, 2-methyl- | 74.07 |
| 7.325 | 10.0 | | Furan, 4-methyl-2-propyl- | 124.09 |
| 7.481 | 6.7 | | Undecane, 4,4,-dimethyl- | 184.22 |
| 7.648 | 13.3 | * | 1-Butanol, 3-methyl-, acetate | 130.10 |
| 7.836 | 18.3 | * | 2-n-Butyl furan | 124.09 |
| 8.026 | 10.8 | + | Benzene, 1,3-dimethyl- [symbol] | 106.08 |
| 8.114 | 12.2 | | Decane, 3,3,5-trimethyl- | 184.22 |
| 8.303 | 11.4 | * | Pentane, 1-iodo- | 197.99 |
| 8.364 | 8.7 | * | 2-Hexanol | 102.10 |
| 8.498 | 7.4 | * | Acetic acid, pentyl ester | 130.10 |
| 8.735 | 14.9 | * | Hexanoic acid, methyl ester | 130.10 |
| 9.066 | 11.5 | * | 1-Butanol, 3-methyl- | 88.09 |
| 9.302 | 8.1 | | Phenol, 4-ethyl- | 122.07 |
| 9.817 | 12.2 | * | 3-Octanone | 128.12 |
| 10.054 | 11.4 | * | Acetic acid, hexyl ester | 144.12 |
| 10.708 | 5.4 | * | 2-Heptanol | 116.12 |
| 10.985 | 14.9 | | 7-Octen-2-one | 126.10 |
| 11.242 | 12.1 | | Cyclopropane, propyl- | 84.09 |
| 11.329 | 9.5 | * | Acetic acid, sec-octyl ester | 172.15 |
| 11.545 | 17.9 | * | Acetic acid, heptyl ester | 158.13 |
| 11.775 | 7.4 | * | Octanoic acid, methyl ester | 158.13 |
| 11.938 | 9.5 | | 3,5-Octadiene (Z, Z) | 110.11 |
| 12.127 | 9.4 | * | 2-Octanol | 130.14 |
| 12.878 | 9.4 | * | Acetic acid | 60.02 |
| 12.931 | 22.7 | * | Acetic acid, octyl ester | 172.15 |
| 13.381 | 4.1 | | 3-Octen-2-ol (Z) | 128.12 |
| 13.584 | 7.3 | | Neoisolongifolene | 204.19 |
| 14.455 | 8.8 | * | Propanoic acid, 2-methyl- | 88.05 |
| 14.543 | 8.8 | | Cycloheptanone, 2-methylene- | 124.09 |
| 14.651 | 12.9 | | 1H-Indene, octahydro-, cis | 124.13 |
| 14.926 | 8.1 | | Cyclopentadiene, 5,5-dimethyl-1-ethyl- | 122.11 |
| 15.335 | 10.1 | * | Butyrolactone | 86.04 |
| 15.477 | 8.1 | * | Acetic acid, decyl ester | 200.18 |
| 15.673 | 13.5 | | Pentanoic acid, 3-methyl- | 116.08 |
| 15.835 | 6.8 | | Cyclodecene | 138.14 |
| 16.472 | 12.7 | | Pentanoic acid | 102.07 |
| 17.653 | 16.9 | * | Hexanoic acid | 116.08 |
| 18.360 | 13.6 | * | Phenylethyl alcohol | 122.07 |
| 19.588 | 8.0 | * | Phenol, 4-ethyl-2methoxy- | 152.08 |

Compounds found fin the control oatmeal agar bottle are not included in this table. Comparative GC/MS data/notes with standard compounds are indicated in the footnotes under - “stds.” The total dry weight of the mycelial mat under these conditions was 38.9 mg.
* Denotes that the retention time and MS spectrum closely matched or were identical to an authentic standard compound. Those compounds without a designated footnote have a MS spectrum that most closely matched the appropriate compound in the NIST data base.
+ Denotes that the compound, as detected in diesel fuel, was used as a standard.
♦ Indicates that the retention time of the standard compound does not match that of the fungal product. It is to be noted that sometimes retention times of a compound can be influenced by other compounds in the injection mixture. Alternatively, the designated compound may represent an isomer of that compound. [symbol]
Denotes that the compound was detected in Patagonian diesel.

There are many more compounds found in the microaerophilically grown Gliocladium sp. on oatmeal than on regular PDA in a Petri plate (e.g., as illustrated by comparing Table 7 to Table 4). Furthermore, many of these compounds are hydrocarbons of great interest to those involved in bio-energy production, especially those alkanes, alkenes, and cyclic alkanes and alkenes. A few compounds found in the PDA growth condition were not observable in the gases of the fungus grown on oatmeal agar under microaerophilic conditions; these include the 1,3,5,7,-cyclooctatetraene and a few others.

### Example 7

### Production of Hydrocarbons by Gliocladium sp. Under Microaerophilic Conditions on Cellulose-Based Medium G

### Cellulose 25 g/L Plus Salts plus 0.1 mg/L of Vitamins B1, B3 and B6

The dramatic increase in global food prices spurs the need to sever the biofuel market from food production. An attractive carbon source is cellulose, the world's most abundant natural organic compound which is digestible by many rainforest fungi including those closely related to G. roseum, namely Trichoderma spp. G. roseum grew for several months on a plate of Cellulose (25 g/L) with salts and yeast extract (0.1 g/L) (cellulose-base media) and it remained inhibitory to P. ultimum (the salts and agar concentration used in this medium followed a recipe of the M1-D medium previously outlined by Pinkerton, F. and Strobel, G. A. (1976) Proc Natl Acad Sci (USA) 73, 4007-4011. Gas analysis after 18 days of incubation on Oatmeal Agar, showed the presence of a number of hydrocarbons commonly found in diesel fuel in the air space of this culture. These included such compounds as heptane, octane, and dodecane (Table 7). Other hydrocarbons were also present including cyclopentane, 1,1,3,3, tetramethyl- and hexane, 3,3,-dimethyl; decane, 2,6,7-trimethyl. Also, the alkane esters present in gases from G. roseum grown on oatmeal-based medium were not found in the volatiles from cellulose based media, however, there were a number of alkane-based free alcohols including 1-decanol, 1-butanol, 2-hexanol, and 1-heptanol. Furthermore, a number of other oxygenated hydrocarbons appeared as ketones such as 3-hexanone; 3-hexanone, 4-methyl; and pentanone, 4-methyl which was the most abundant volatile product in this mixture (Table 8). Each of these volatile hydrocarbons is readily combustible. Both ethanol and acetic acid were present in Cellulose based media as they were in Oatmeal based media along with some sesquiterpenoids including aciphyllene, eremophilene, and others (Tables 7 and 8).

TABLE 8
A GC/MS air space analysis of the volatile compounds produced by G. roseum after an
18 day incubation under microaerophilic conditions at 23° C. on cellulose-base media

| Time | | | Possible | Molecular |
|---|---|---|---|---|
| (min) | Relative Area | Stds *+♦ | Compound [symbol] | Weight |
| 1.607 | 97.511 | | Cyclopropane, 1,2-dimethyl-cis- | 70.08 |
| 1.922 | 13.783 | *+ | Heptane [symbol] | 100.12 |
| 2.608 | 1.830 | *+ | Octane [symbol] | 114.14 |
| 3.610 | 2.048 | | Cyclopentane,1,1,3,3-tetramethyl- | 126.14 |
| 4.562 | 50.710 | * | Ethanol | 46.04 |
| 4.670 | 5.547 | * | Benzene | 78.05 |
| 5.248 | 0.473 | * | 2-Pentanone | 86.07 |
| 5.333 | 1.371 | ♦ | Hexane, 3,3-dimethyl-(or isomer) | 114.14 |
| 5.408 | 1.795 | | Decane, 2,6,7-trimethyl- | 184.22 |
| 5.747 | 397.632 | * | Pentanone, 4-methyl- | 100.09 |
| 5.820 | 40.064 | * | 1-Propen-2-ol, acetate | 100.05 |
| 5.968 | 2.632 | ♦ | Hexadecane (or isomer) [symbol] | 226.26 |
| 6.492 | 1.577 | * | 3-Hexanone | 100.09 |
| 6.853 | 4.860 | | 3-Hexanone, 4-methyl- | 114.10 |
| 6.976 | 1.102 | *+ | Dodecane [symbol] | 170.20 |
| 7.130 | 1.441 | ♦ | 1-Decanol (or isomer) | 158.17 |
| 7.198 | 2.365 | | Cyclopentanone, 2,4-dimethyl- | 112.09 |
| 7.903 | 0.919 | ♦ | 2-Pentene, 2,3-dimethyl-(or isomer) | 98.11 |
| 8.027 | 1.718 | * | 1-Butanol | 74.07 |
| 8.333 | 28.715 | * | 2-Hexanol | 102.10 |
| 8.675 | 1.430 | | Hexane, 2,3,4-trimethyl- | 128.16 |
| 9.013 | 47.600 | * | 1-Butanol, 3-methyl-(impure) | 88.09 |
| 9.291 | 2.850 | | Undecane 4,7-dimethyl- | 184.22 |
| 9.422 | 2.111 | * | Furan, 2-pentyl- | 138.10 |
| 10.138 | 1.617 | *+ | Nonadecane [symbol] | 268.31 |
| 10.298 | 1.624 | * | Benzene, 1,2,3-trimethyl- | 120.09 |
| 10.437 | 6.675 | | 3-Heptanone, 5-ethyl-4-methyl- | 156.15 |
| 10.628 | 1.253 | | Octadecane, 1-chloro- | 288.26 |
| 11.190 | 1.444 | * | 1-Hexanol | 102.10 |
| 11.330 | 1.475 | * | Benzene, bromo- | 155.96 |
| 11.775 | 0.858 | * | 2-Nonanone | 142.13 |
| 11.912 | 1.070 | | Nonanoic acid, 2,4-dimethyl, methyl ester | 200.18 |
| 12.605 | 1.662 | * | 1-Heptanol | 116.12 |
| 12.719 | 4.415 | * | 2-Propanone, methylhydrazone | 86.08 |
| 12.809 | 26.776 | * | Acetic acid | 60.02 |
| 13.556 | 4.539 | | unknown | 204.19 |
| 14.213 | 7.333 | * | α-Gurjunene | 204.19 |
| 14.598 | 4.076 | | Aciphyllene | 204.19 |
| 14.648 | 1.223 | | Δ-Guaiene | 204.19 |
| 14.902 | 24.840 | * | Benzonitrile | 103.04 |
| 14.957 | 3.169 | | (−) β-Lepidozene | 204.19 |
| 15.522 | 3.996 | | Eremophilene | 204.19 |
| 15.961 | 1.625 | * | α-Humulene | 204.19 |
| 16.037 | 1.478 | ♦ | Camphene (or isomer) | 136.12 |
| 16.104 | 3.081 | * | Benzene, 1,4-dibromo | 233.87 |
| 17.281 | 6.366 | | Benzene, 1,3,5-trichloro-2-methoxy- | 209.94 |
| 17.840 | 0.430 | | 6-Methoxy-1-acetonaphthone | 200.08 |
| 17.905 | 0.570 | * | Benzenemethanol | 108.06 |
| 18.596 | 2.401 | * | Benzeneethanol | 122.07 |

Compounds found in the control bottle are not included in this table. Comparative GC/MS data/notes with standard compounds are indicated in the footnotes under - “Stds.” The total dry weight of the mycelial mat under these conditions was 4.7 mg.
* Denotes that the retention time and MS spectrum closely matched or were identical to an authentic standard compound. Those compounds without a designated footnote have a MS spectrum that most closely matched to the appropriate compound in the NIST data base.
+ Denotes that the compound, as detected in diesel fuel that was used as a standard.
♦ Indicates that the retention time of the standard compound does not match that of the fungal product. It is to be noted that sometimes retention times of a compound can be strongly influenced by other compounds in the injection mixture. Alternatively, the designated compound may represent an isomer of that compound. [symbol]
Denotes that the compound was detected in Patagonian diesel.

This fungus does make other lipids in addition to those hydrocarbons appearing in the gas phase. For instance, the methylene chloride extract of the cellulose-based medium, after 30 days of incubation, yielded at least 130 mg of lipoidal substances. Thin layer chromatography in solvents A, B and C, with co-chromatography of appropriate standards, indicated the presence of linoleic acid, linolenic acid combined with a mixture of triglycerides, and one or more cholic acid derivatives. Only trace amounts of lipoidal compounds were detected in the control by comparable methods. This suggests that, in addition to the volatiles of this fungus being important and useful, so too are components in the aqueous phase.

### Example 8

### Production of Hydrocarbons and their Derivatives by G. roseum Under Microaerophilic Conditions on “Ulmo” Medium

Ulmo medium, made from the host plant extract of G. roseum, was tested for the production of volatiles. Eucryphia cordifolia “ulmo” stem medium is produced by boiling 30 g of newly developing stem tissues in water for 30 min, filtering and then adding water to 1 L. The salts and agar concentration used in this medium followed a recipe of the M1-D medium previously outlined by Pinkerton, F. and Strobel, G. A. (1976) Proc Natl Acad Sci (USA) 73, 4007-4011.

This medium was tested to determine if the host plant from which the fungus was originally isolated could both support fungal growth and simultaneous production of a volatile mixture possessing alkanes or other hydrocarbons. It was surmised that the microaerophilic conditions in the test circumstances mimicked those that this endophytic fungus may experience in the host tissue in terms of nutrient and oxygen availability, since endophytes are located in the intercellular spaces of plant cells. Interestingly, the organism produced several alkanes and an alkene (diesel hydrocarbons) in the volatiles detected in the air space of the 18 day old culture (Table 9). These included octene; undecane, 2,7, -dimethyl; decane, 4-methyl; and tridecane (Table 9). Numerous other alkanes were also produced along with several sesquiterpenoids (Table 9). Since the volatiles of this fungus possess antibiotic properties, these compounds may play a role protecting the plant from invading pathogens (Ezra, D. et al., (2004) Plant Sci, 166, 1471-1477.

TABLE 9
A GC/MS air space analysis of the volatile compounds produced
by G. roseum after an 18 day incubation under microaerophilic
conditions at 23° C. on Ulmo medium

| Time | Relative | | | Molecular |
|---|---|---|---|---|
| (min) | Area | Stds *+ | Possible Compound [symbol] | Weight |
| 1.552 | 14.136 | * | 1-Butene, 2-methyl- | 70.08 |
| 1.816 | 0.743 | * | Heptene | 100.12 |
| 2.806 | 2.304 | *+ | 1-Octene [symbol] | 112.12 |
| 3.572 | 0.992 | | Furan, 2-methyl- | 82.04 |
| 4.385 | 5.389 | | 1,3-Cyclopentadiene, 1,2- | 94.08 |
| | | | dimethyl- | |
| 4.789 | 1.071 | + | Dodecane, 4,6-dimethyl- [symbol] | 198.23 |
| 4.850 | 1.933 | | Undecane, 4,6-dimethyl- | 184.22 |
| 5.369 | 3.074 | | Undecane, 5-methyl- | 170.20 |
| 6.110 | 0.845 | | Nonane, 5-propyl- | 170.20 |
| 6.171 | 5.051 | + | Undecane, 4-methyl- [symbol] | 170.20 |
| 6.246 | 2.394 | | Undecane, 2,7-dimethyl- | 184.22 |
| 6.396 | 2.879 | + | Decane, 4-methyl- [symbol] | 156.19 |
| 6.546 | 1.447 | | 1-Decane, 8-methyl- | 154.17 |
| 8.407 | 2.114 | * | 1-Butanol, 2-methyl- | 88.09 |
| 8.625 | 0.984 | *+ | Tridecane [symbol] | 184.22 |
| 9.484 | 1.314 | | Dodecane, 2,7,10-trimethyl- | 212.25 |
| 11.591 | 11.384 | * | Benzene, 1-methoxy-2-methyl- | 122.07 |
| 12.075 | 1.046 | * | Benzene, 1-methoxy-3-methyl- | 122.07 |
| 12.847 | 2.816 | | 1,3-cyclopentadiene, 1,2,3,4,5- | 136.12 |
| | | | pentamethyl- | |
| 14.292 | 4.192 | | Phenol, 3-ethyl- | 122.07 |
| 14.770 | 2.669 | | Pinocarveol | 152.12 |
| 14.927 | 0.900 | * | (+)-Aromadendrene | 204.19 |
| 15.367 | 0.871 | * | α-Humulene | 204.19 |
| 16.324 | 2.829 | * | Myrtenol | 152.12 |
| 16.691 | 1.006 | | Benzene, 1,3,5-trichloro-2- | 209.94 |
| | | | methoxy- | |
| 20.292 | 0.817 | ♦ | Phenol, 4-ethyl- | 122.07 |

Compounds found in the control bottle are not included in this table. Comparative GC/MS data/notes with standard compounds are indicated in the footnotes under - “Stds.” The total dry weight of the mycelial mat under these conditions was 5.3 mg.
* Denotes that the retention time and MS spectrum closely matched or were identical to an authentic standard compound. Those compounds without a designated footnote have a MS spectrum that most closely matched to the appropriate compound in the NIST data base.
+ Denotes that the compound, as detected in diesel fuel that was used was used as a standard.
♦ Indicates that the retention time of the standard compound does not match that of the fungal product. It is to be noted that sometimes retention times of a compound can be strongly influenced by other compounds in the injection mixture. Alternatively, the designated compound may represent an isomer of that compound. [symbol]
Denotes that the compound was detected in Patagonian diesel.

### Example 9

### Quantification of Volatile Products of G. Roseum by PTR-MS

Airspace analysis of the cultured and uninoculated samples was determined by flowing a small flow of air (medical grade compressed air) through the culture bottles and then diluting this gas with catalytically scrubbed (to remove hydrocarbons) room air. Measurements were made on an 18 day old culture of G. roseum grown on a 300 ml oatmeal agar slant in a 950 ml brown glass bottle at 23° C. with a septum that had been modified to possess both inlet and outlet tubes (Ezra, D., et al. (2004) Plant Sci., 166, 1471-1477). The tests were run with a drift tube temperature of 30° C. with 10 mL/min of air space air being diluted into 90 mL/min of hydrocarbon-free room air. The 1/10 dilution kept the measurements within the linear dynamic range of the instrument and kept water from condensing in the sample lines. The sample lines were constructed totally from PFA Teflon tubing and fittings. Mass spectral scans were acquired from 20-250 Daltons. A minimum of 10 mass spectral scans were obtained for each sample. Concentrations derived from the PTR-MS measurements were calculated using equations derived from reaction kinetics and assume a reaction rate coefficient of 2×10⁻⁹ mL/s is appropriate for all compounds (Lindinger, W., et al., (1998) Internat. J. Mass Spectrometry Ion. Proc., 173, 191-241; Ezra, D., et al. (2004) Plant Sci., 166, 1471-1477). This method provides a simple means through which the measured ion intensity at any mass can be expressed as an equivalent concentration. In the event that a particular ion can be ascribed to a singular compound, then the concentration of that specific compound can be determined using the same procedure as above followed by correction for any product ion fragmentation. The product ion distribution is determined from mixtures prepared from pure standards.

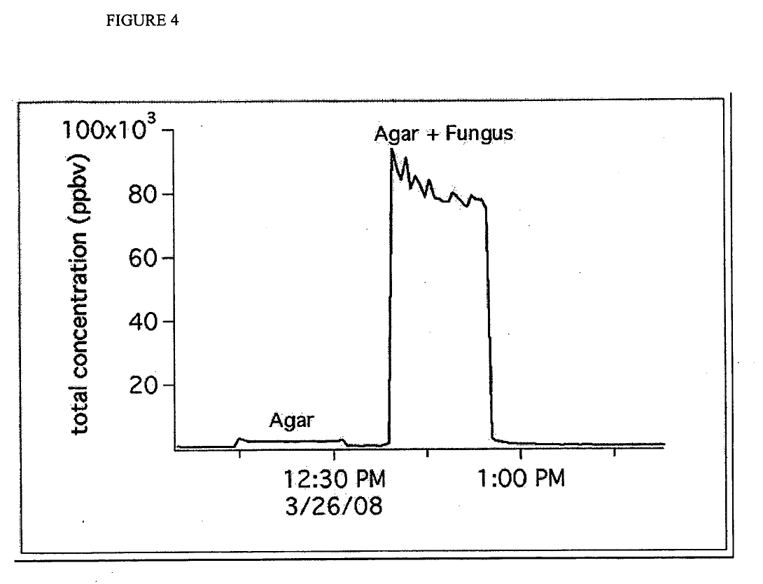

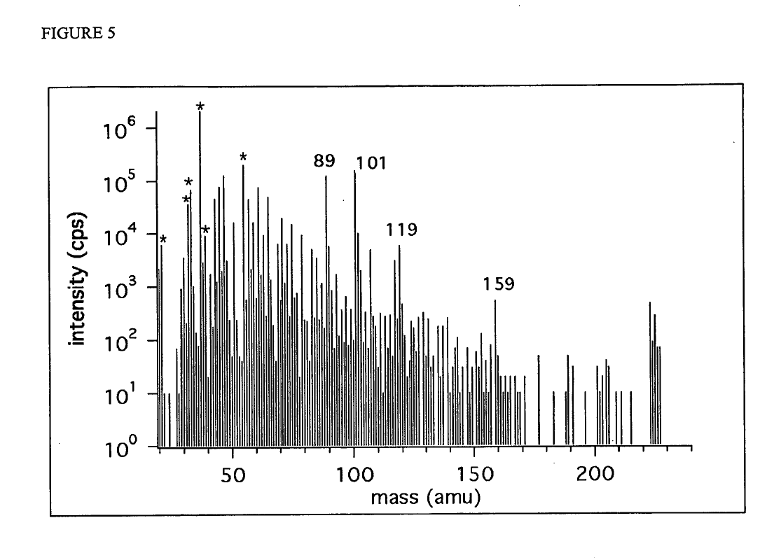

The total concentration of volatile organic compounds produced by the fungus in Oatmeal medium and detectable by the PTR-MS was estimated to be in the order of 80 ppmv with the contribution of the control agar gases being excluded (FIG. 4). This value was calculated by summing up the concentration from each ion in the mass spectrum (FIG. 5). Ions that were deemed as reagent ions, contaminant ions such as O₂+ or NO+, or water-related ions, were not included in this calculation. Additional major contributors to the total volatiles were methanol, ethanol and acetaldehyde ions making up about 30-40% of the total. Also, major contributors were ions at m/z 89 and 101. The identity of these two ions could not be made on the basis of the compounds shown in Table 7, but the ion at m/z 89 is consistent with protonated butyric acid ion. Overall, it seems that compounds, in addition to those shown in Table 7, are also produced by this fungus. This is not unexpected given the fact that the microextraction fiber (Stable Flex™) does not universally absorb all volatile compounds.

Also, in order to quantify the concentration of the hydrocarbons, and their derivatives, present in the air space of medium A, an indirect method involving PTR-MS was used. One of the most abundant volatiles is acetic acid heptyl ester with a retention time of 11.545 min (Table 7) and its protonated molecule is detected at m/z 159 by the PTR-MS. The concentration of the acetic acid heptyl ester has been quantified assuming all of the intensity at m/z 159 originated from this compound. The peak concentration for this compound was on the order of 500 ppbv. Assuming that the other volatile compounds are present in the air space vapor in the same proportions determined by the GC/MS, then the concentration of the acetic acid heptyl ester can be used to estimate the concentrations of all other ingredients in the volatile phase (Table 7). The total concentration of volatiles in the air space of medium A was estimated at 4 ppmv. It is to be noted, however, that certain volatile compounds may not have adsorbed to the fiber and others may have adsorbed in a uniform manner, thus yielding a lower than expected total amount of material in the gas phase as calculated by this indirect method versus the concentration of volatile compounds based on total ion yield (above). In addition, the concentration of volatiles found in medium G was calculated by this indirect method and shown to be in the range of 50% of that in medium A.

These findings demonstrate that under microaerophilic conditions, Gliocladium sp. produces several energy rich substances including a selection of volatile alkanes and alkenes. Such findings are expected to be highly significant to the biofuel industry because of the need for more sources of bio-energy.

### Example 10

### Methods for Isolating the Genome Sequence of Isolate C-13

This example provides representative methods for isolating genomic sequence, including the complete genome sequence, of Gliocladium isolate C-13.

A whole genome random sequencing method (Fleischmann et al., Science 269:496, 1995; Fraser et al., Science 270:397, 1995) can be used to obtain the complete genome sequence for Gliocladium isolate C-13. A small insert plasmid library (2.5 Kbp average insert size) and a large insert lambda library (16 Kbp average insert size) can be used as substrates for sequencing. The lambda library can be used to form a genome scaffold and to verify the orientation and integrity of the contigs formed from the assembly of sequences from the plasmid library. All clones can be sequenced from both ends to aid in ordering of contigs during the sequence assembly process. In an example, the average length of sequencing reads 481 base pairs. The sequences can be assembled by means of the TIGR Assembler (Fleischmann et al., Science 269:496 (1995); Fraser et al., Science 270:397 (1995); Sutton et al., Genome Sci. Tech. 1:9, 1995). Sequence and physical gaps can be closed using a combination of strategies (Fleischmann et al., Science 269:496, 1995; Fraser et al., Science 270:397, 1995). The colinearity of the in vivo genome to the genome sequence can be confirmed by comparing restriction fragments from six, rare cutter, restriction enzymes (such as Aat II, BamHI, Bgl II, Kpn I, Sma I, and Sst II) to those predicted from the sequence data. Additional confidence in the colinearity can be provided by the genome scaffold produced by sequence pairs from large-insert lambda clones, which covered the majority (such as 85%) of the main chromosome. Open reading frames (ORFs) and predicted protein-coding regions can be identified as previously described (Fleischmann et al., Science 269:496, 1995; Fraser et al., Science 270:397, 1995). The predicted protein-coding regions can be searched against the Blocks database (Henikoff & Henikoff, Genomics 19:97, 1994) by means of BLIMPS (Wallace & Henikoff, CABIOS 8:249, 1992) to verify putative identifications and to identify potential functional motifs in predicted protein-coding regions that have no database match. Hydrophobicity plots can be performed on all predicted protein-coding regions by means of the Kyte-Doolittle algorithm (Kyte & Doolittle, J. Mol. Biol. 157:105, 1982) to identify potentially functionally relevant signatures in these sequences.

### Example 11

### Methods of Creating a Genomic DNA Library

This example provides representative, non-limiting methods for producing a Gliocladium isolate C-13 genomic DNA library. Gliocladium isolate C-13 hydrocarbon production related DNA can be obtained by screening a genomic DNA library.

The choice of methods for creating a genomic DNA library are not limited, and any suitable method may be used, preferably being a general method for constructing a genomic DNA library of a eukaryotic organism. Examples thereof include the method of by Sambrook et al. (Molecular Cloning: A Laboratory Manual, 2nd Ed., Vols. 1-3, Cold Spring Harbor Press (1989)). Other suitable methods are known in the art.

In outline, genomic DNA can be obtained by recovering cells from a culture of Gliocladium isolate C-13, physically breaking the cells, extracting DNA present in the nuclei thereof and purifying said DNA. Culturing of Gliocladium isolate C-13 can be performed under conditions suitable for Gliocladium isolate C-13 (including those described in Examples 1, 3, 6, 7, and 8). Cells of an ML-236B producing micro-organism cultured in a liquid medium can be recovered by centrifugation, and those cultured on a solid medium can be recovered by scraping from the solid media with a cell scraper or the like.

Physical breaking of cells can be performed by grinding the cells using a pestle and a mortar, after freezing them with liquid nitrogen or the like. DNA in the nuclei of the broken cell can be extracted using a surfactant such as sodium dodecyl sulfate (SDS) or other suitable surfactant. The extracted genomic DNA is suitably treated with phenol-chloroform to remove protein, and recovered as a precipitate by performing an ethanol precipitation.

The resulting genomic DNA is fragmented by digestion with a suitable restriction enzyme. There is no limitation on the restriction enzymes that can be used for the restriction digest, with generally available restriction enzymes preferred. Examples thereof include Sau3AI. Other suitable enzymes are known in the art. Digested DNA is then subjected to gel electrophoresis, and genomic DNA having a suitable size is recovered from the gel. The size of DNA fragment is not particularly limited, but is may be 20 kb or more.

There is likewise no limitation on the choice of DNA vector used in construction of the genomic DNA library as long as the vector has a DNA sequence necessary for replication in the host cell which is to be transformed by the vector. Examples of suitable vectors include a plasmid vector, a phage vector, a cosmid vector, a BAC vector or the like, with a cosmid vector being preferred. The DNA vector is preferably an expression vector. More preferably, the DNA vector comprises a DNA or nucleotide sequence which confers a selective phenotype onto the host cell transformed by the vector.

The DNA vector is suitably a vector that can be used in both cloning and expression. Preferably the vector is a shuttle vector which can be used for transformation of more than one micro-organism host. The shuttle vector suitably has a DNA sequence which permits replication in a host cell, and preferably a sequence or sequences which permit replication in a number of different host cells from different micro-organism groups such as bacteria and fungi. Furthermore, the shuttle vector preferably comprises a DNA sequence which can provides a selectable phenotype for a range of different host cells, such as cells from different micro-organism groups.

The choice of combination of a micro-organism groups and host cells transformed by the shuttle vector is not particularly limited, provided that one of the micro-organism groups can be used in cloning and the other has Gliocladium isolate C-13 producing ability. Such combination can be, for example, a combination of a bacterium and fungi or a combination of yeast and fungi. The choice of bacterium is not particularly limited as long as it can be generally used in biotechnology, such as for example Escherichia coli, Bacillus subtilis or the like. Similarly there is no restriction on yeast species as long as it can be generally used in biotechnology, such as for example, Saccharomyces cerevisiae or the like.

Examples of the above-mentioned shuttle vector include a cosmid vector having a suitable marker gene for selecting a phenotype and a cos site. Other suitable vectors are known in the art. A genomic DNA library can be prepared by introducing a shuttle vector into a host cell, the vector containing a genomic DNA fragment from a Gliocladium isolate C-13. The host cell to be used may be E. coli, for instance E. coli XL1-Blue MR. When the host cell is E. coli, introduction can be performed by in vitro packaging.

A genomic library can be screened to identify a desired clone using an antibody or a nucleic acid probe, with a nucleic acid probe being preferred. Suitable nucleic acid probes can be obtained, for example, by synthesizing an oligonucleotide probe comprising part of a known genomic DNA sequence as described above, or by preparing oligonucleotide primers and amplifying the target DNA using the polymerase chain reaction and genomic DNA as a template, or by RT-PCR using mRNA as a template. Other suitable methods for obtaining such probes are well known in the art.

### Example 12

### Methods of Isolating and Cloning Isolate C-13 Nucleic Acid Molecules

This example provides methods for isolating and cloning nucleic acids from the genome of Gliocladium isolate C-13, such as that obtained using methods described in Examples 10 and 11. It will be recognized that, with the provision of a deposit of this isolate, sequencing and cloning of individual genes within the Gliocladium isolate C-13 genome is now enabled. Also enabled are methods of employing one or more sequence from this genome to engineer a target organism, thereby imparting to that organism the ability (or an enhanced ability) to produce compounds useful in the production of biofuels. Also enabled now are methods for expressing individual proteins from genes in Gliocladium isolate C-13, for instance to be used in vitro alone or in combination in order to produce compounds useful in the production of biofuels.

### Preparation of PCR Primers and Amplification of DNA.

Various fragments of the Gliocladium isolate C-13 genome can be used to prepare PCR primers. The PCR primers are preferably at least 15 bases, and more preferably at least 18 bases in length. When selecting a primer sequence, it is preferred that the primer pairs have approximately the same G/C ratio, so that melting temperatures are approximately the same. The PCR primers are useful during PCR cloning of the ORFs, such as individual ORFs from the organism described herein.

Gene Expression from DNA Sequences Corresponding to ORFs.

A fragment of the Gliocladium isolate C-13 genome (for instance, a protein-encoding sequence) is introduced into an expression vector using conventional technology (techniques to transfer cloned sequences into expression vectors that direct protein translation in mammalian, yeast, insect or bacterial expression systems are well known in the art). Commercially available vectors and expression systems are available from a variety of suppliers including Stratagene (La Jolla, Calif.), Promega (Madison, Wis.), and Invitrogen (San Diego, Calif.). If desired, to enhance expression and facilitate proper protein folding, the codon context and codon pairing of the sequence may be optimized for the particular expression organism, as explained by Hatfield et al., U.S. Pat. No. 5,082,767, which is hereby incorporated by reference.

The following is provided as one exemplary method to generate polypeptide(s) from a cloned ORF of the Gliocladium isolate C-13 genome. A polyA sequence can be added to the construct by, for example, splicing out the poly A sequence from pSGS (Stratagene) using BglII and SalI restriction endonuclease enzymes and incorporating it into the mammalian expression vector pXT1 (Stratagene) for use in eukaryotic expression systems. pXT1 contains the LTRs and a portion of the gag gene from Moloney Murine Leukemia Virus. The position of the LTRs in the construct allows efficient stable transfection. The vector includes the Herpes Simplex thymidine kinase promoter and the selectable neomycin gene. The isolate C-13 DNA is obtained by PCR from the bacterial vector using oligonucleotide primers complementary to the isolate C-13 DNA and containing restriction endonuclease sequences for PstI incorporated into the 5′ primer and BglII at the 5′ end of the corresponding Gliocladium isolate C-13 DNA 3′ primer, taking care to ensure that the isolated Gliocladium isolate C-13 DNA is positioned such that its followed with the polyA sequence. The purified fragment obtained from the resulting PCR reaction is digested with PstI, blunt ended with an exonuclease, digested with BglII, purified and ligated to pXT1, now containing a polyA sequence and digested BglII.

The ligated product can be transfected into mouse NIH 3T3 cells using Lipofectin (Life Technologies, Inc., Grand Island, N.Y.) under conditions outlined in the product specification. Positive transfectants are selected after growing the transfected cells in 600 μg/ml G418 (Sigma, St. Louis, Mo.). The protein is released into the supernatant, retained within the cell or expression may be restricted to the cell surface.

Methods for expressing large amounts of protein from a cloned gene introduced into a heterologous expression system, such as E. coli, are utilized for the purification and functional analysis of proteins. Methods and plasmid vectors for producing proteins in bacteria (or other heterologous expression systems) are described by Sambrook et al. ((ed.), Molecular Cloning: A Laboratory Manual, 2^{nd} ed., vol. 1-3, Cold Spring Harbor Laboratory Press, Cold Spring Harbor, N.Y., 1989). Isolation and purification of recombinantly expressed proteins are carried out by conventional means including preparative chromatography and immunological separations.

In view of the many possible embodiments to which the principles of the disclosed invention may be applied, it should be recognized that the illustrated embodiments are only examples and should not be taken as limiting the scope of the invention.

All publications, patents and patent applications herein are incorporated by reference to the same extent as if each individual publication or patent application was specifically and individually indicated to be incorporated by reference.

The foregoing detailed description has been given for clearness of understanding only and no unnecessary limitations should be understood therefrom as modifications will be obvious to those skilled in the art. It is not an admission that any of the information provided herein is prior art or relevant to the presently claimed inventions, or that any publication specifically or implicitly referenced is prior art.

Unless defined otherwise, all technical and scientific terms used herein have the same meaning as commonly understood by one of ordinary skill in the art to which this invention belongs.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

We claim:

1. An isolated strain of a Gliocladium spp., wherein the strain is Gliocladium isolate C-13 (deposited as NRRL 50072).

2. A method for producing one or more volatile organic compounds (VOCs), comprising culturing Gliocladium isolate C-13 (NRRL 50072) under conditions sufficient for producing VOCs.

3. The method of claim 2, wherein the conditions sufficient for producing VOCs comprise culturing Gliocladium isolate C-13 (NRRL 50072) in culture medium comprising oatmeal agar.

4. The method of claim 3, wherein under conditions sufficient for producing VOCs further comprise culturing Gliocladium isolate C-13 (NRRL 50072) in microaerophilic conditions.

5. The method of claim 4, wherein said conditions comprise culturing Gliocladium isolate C-13 (NRRL 50072) in a bioreactor vessel.

6. The method of claim 5, wherein said bioreactor vessel has a volume from about 100 ml to about 10,000 L or larger.

7. The method of claim 5, further comprising isolating a VOC from the culture medium or from the vapor in the vessel.

8. The method of claim 7, wherein said isolating a VOC comprises fractional distillation and/or absorption chromatography.

9. The method of claim 2, wherein the VOC is an alkane, an alkene, an alkyne, a diene, an isoprene, an alcohol, an aldehyde, a carboxylic acid, a wax ester, or a mixture of any two or more thereof.

10. The method of claim 2, wherein said VOC is selected from Table 4, 7, 8, or 9.

11. The method of claim 10, wherein said VOC can be used to produce biofuel, jet fuel, plastics, plasticizers, antibiotics, rubber, fuel additives, and/or adhesives.

12. A kit for making one or more VOCs comprising Gliocladium spp. and instructions for growing said Gliocladium spp. under optimal conditions for optimal VOC production, wherein the Gliocladium spp. is Gliocladium isolate C-13 (deposited as NRRL 50072).

13. The kit of claim 12, further comprising growth media.

14. The kit of claim 12, wherein said media is an oatmeal based media.

15. A method for producing volatile organic compounds (VOC), comprising culturing an anamorph of Ascocoryne spp. under conditions sufficient for producing VOC.

16. The method according to claim 16, wherein said anamorph is Gliocladium isolate C-13 (NRRL 50072).

17. An isolated nucleic acid molecule from Gliocladium isolate C-13 (NRRL 50072) encoding a polypeptide involved in the synthesis or production of one or more VOCs.

18. The isolated nucleic acid molecule of claim 17, wherein said VOC is selected from those listed in Tables 4, 7, 8, or 9.

19. A heterologous organism comprising said isolated nucleic acid molecule of claim 17, wherein said isolated nucleic acid molecules encodes a polypeptide involved in the synthesis or production of one or more VOCs.

20. The heterologous organism of claim 19, wherein said VOC is selected from those listed in Tables 4, 7, 8, or 9.
